(19) United States
(12) Patent Application Publication
(10) Pub. No.: US 2022/0105269 A1
(43) Pub. Date: Apr. 7, 2022

(54) AUTOMATED PROJECTION MODE SWITCHING FOR GLUCOSE LEVELS

(71) Applicant: Medtronic MiniMed, Inc., Northridge, CA (US)

(72) Inventors: Yuxiang Zhong, Arcadia, CA (US); Pratik J. Agrawal, Porter Ranch, CA (US); Chantal M. McMahon, Atlanta, GA (US); Dae Kang, Los Angeles, CA (US); David Legray, Northridge, CA (US); Nicole T. Robinson, Sherman Oaks, CA (US); Natassia Mattoon, La Mirada, CA (US); Michael A. Hill, South Pasadena, CA (US)

(21) Appl. No.: 17/481,745

(22) Filed: Sep. 22, 2021

Related U.S. Application Data
(60) Provisional application No. 63/086,718, filed on Oct. 2, 2020.

(51) Int. Cl. A61M 5/172; A61M 5/142; G16H 20/17

(52) U.S. Cl. CPC A61M 5/1723; A61M 5/14244; G16H 20/17; A61M 2205/583; A61M 2205/18; A61M 2005/14208; A61M 2205/52

(57) ABSTRACT

In general, techniques are described for automated projection mode for switching glucose levels. A device for assisting in therapy delivery comprising a memory and a processor configured to perform the techniques. The memory may store first projected levels of glucose in a patient over a first time frame. The processor may determine an occurrence of a projection event that alters how projected levels of glucose are to be output, and automatically determine, based on the projection event, a second time frame that differs from the first time frame, The processor may also obtain a current glucose level of the patient, obtain, based on the current glucose level, second projected levels of glucose in the patient over the second time frame, and output the second projected levels of glucose for the second time frame.

This application claims the benefit of U.S. Provisional Application No. 63/086,718, filed Oct. 2, 2020, the entire contents of which are incorporated by reference herein.

### TECHNICAL FIELD

The disclosure relates to medical systems and, more particularly, to medical systems directed to therapy for diabetes.

### BACKGROUND

A patient with diabetes receives insulin from a pump or injection device to control the glucose level in his or her bloodstream. Naturally produced insulin may not control the glucose level in the bloodstream of a diabetes patient due to insufficient production of insulin and/or due to insulin resistance. To control the glucose level, a patient's therapy routine may include dosages of basal insulin and bolus insulin. Basal insulin, also called background insulin, tends to keep blood glucose levels at consistent levels during periods of fasting and is a long acting or intermediate acting insulin. Bolus insulin may be taken specifically at or near mealtimes or other times where there may be a relatively fast change in glucose level, and may therefore serve as a short acting or rapid acting form of insulin dosage.

### SUMMARY

Various aspects of techniques for assisting in management of glucose levels in a patient are described. With such techniques, a patient device may automatically dismiss or otherwise disable alerts responsive to detecting a maintenance event that alters projected levels of glucose such that the projected levels of glucose do not leave a prescribed range. That is, the patient device may obtain an indication of various maintenance events, such as a meal event indicative of the patient eating a meal or an insulin injection event in which the patient receives insulin, and update or otherwise revise the projected levels of glucose to reflect the maintenance event. The patient device may then monitor the projected levels of glucose, but in the interim may disable the alert for a temporary period of time (determined based on the revised projected levels of glucose).

By automatically dismissing or otherwise disabling the alert for the temporary period of time, the patient device may avoid needlessly consuming processor cycles, memory bandwidth, memory storage space, or other computing resources that would have otherwise been consumed by repeatedly presenting the alert as a result of not accounting for the maintenance event. Furthermore, by disabling the alert, the patient device may avoid annoying the patient with repeated alerts, which may raise patient engagement with the patient device and thereby improve projections of glucose levels (as the patient may be more willing to enter information regarding insulin delivery, meal consumption, exercise, sleep, and the like).

Moreover, rather than present projected levels of glucose for time frames that are not relevant to the patient in the current context, various aspects of the techniques may enable the patient device to automatically determine projection events that result in dynamic selection of projection modes between the different time frames (e.g., different period of time having different durations of time). For example, the patient device may determine a time of day event (e.g., a meal event, a sleep event, etc. that occurs at a given time of day), a physiological event (e.g., an illness event, a menstruation event, a medication event, etc.), a lifestyle event (e.g., a holiday event, a vacation event, an exercise event, etc.) and/or a data-driven event (e.g., a missing data. event, a prediction inaccuracy event, a historical event, etc.). The patient device may then automatically determine, responsive to the projection event (e.g., without additional user input), a second time frame for which to obtain projected levels of glucose. The patient device may present the revised projected levels of glucose for the determined time frame that better facilitates understanding of the projected levels of glucose in the current context.

As such, by automatically selecting a time frame (e.g., a period of time having a particular duration) by which to project levels of glucose, the patient device may avoid needlessly consuming processor cycles, memory bandwidth, memory storage space, or other computing resources that would have otherwise been consumed by presenting projected levels of glucose that do not account for the current context (as indicated by the projection event). Again, by projecting the glucose levels for the time frame that better fits the current context in which the patient is acting, the patient device may avoid annoying the patient with what would appear to be inaccurate information, which may raise patient engagement with the patient device and thereby improve projections of glucose levels (as the patient may be more willing to enter information regarding insulin delivery, meal consumption, exercise, sleep, and the like).

In one example, the disclosure describes a device for assisting in therapy delivery; the device comprising: a memory configured to store alert data; one or more processors configured to: obtain projected levels of glucose in a patient over a time frame; determine whether the projected levels of glucose leave a prescribed range; generate, when the projected levels of glucose in the patient leave the prescribed range during the time frame and based on the alert data, a graphical alert indicating that the projected. levels of glucose will leave the prescribed range; determine that a maintenance event alters the projected levels of glucose such that the projected levels of glucose do not leave the prescribed range; and disable, without user input and based on the determination that the maintenance event alters the projected levels of glucose such that the projected levels of glucose do riot leave the prescribed range, the graphical alert for a temporary period of time.

In another example, the disclosure describes a method for assisting in therapy delivery, the method comprising: obtaining, by one or more processors, projected levels of glucose in a patient over a time frame; determining, by the one or more processors, whether the projected levels of glucose leave a prescribed range; generating, by the one or more processors_{;} when the projected levels of glucose in the patient leave the prescribed range, and based on alert data, a graphical alert indicating that the projected levels of glucose will leave the prescribed range; determine, by the one or more processors, that a maintenance event alters the projected levels of glucose such that the projected levels of glucose do not leave the prescribed range; and automatically disabling, by the one or more processors and based on the determination that the maintenance event alters the projected levels of glucose such that the projected levels of glucose do not leave the prescribed range, the graphical alert for a temporary period of time.

In another example, the disclosure describes a non.-transitory computer-readable storage medium having instructions stored thereon that, when executed, cause one or more processors to: obtain projected levels of glucose in a patient over a time frame; determine whether the projected levels of glucose leave a prescribed range; generate, when the projected levels of glucose in the patient leave the prescribed range and based on an alert template, a graphical alert indicating that the projected levels of glucose will leave the prescribed range; determine that a maintenance event alters the projected levels of glucose such that the projected levels of glucose do not leave the prescribed range; and automatically disable, based on the determination that the maintenance event alters the projected levels of glucose such that the projected levels of glucose do not leave the prescribed range, the graphical alert for a temporary period of time.

In another example, the disclosure describes a device for assisting in therapy delivery, the device comprising: a memory configured to store first projected levels of glucose in a patient over a first time frame; one or more processors configured to: determine an occurrence of a projection event that alters how projected levels of glucose are to be output; automatically determine, based on the projection event, a second time frame that differs from the first time frame; obtain a current glucose level of the patient; obtain, based on the current glucose level, second projected levels of glucose in the patient over the second time frame; and output the second projected levels of glucose for the second time frame.

In another example, the disclosure describes a method for assisting in therapy delivery, the method comprising: obtaining, by one or more processors, first projected levels of glucose in a patient over a first time frame; determining, by the one or more processors, an occurrence of a projection event that alters how projected levels of glucose are to be output; automatically determining, by the one or more processors and based on the projection event, a second time frame that differs from the first time frame; obtaining, by the one or more processors, a current glucose level of the patient; obtaining, by the one or more processors and based on the current glucose level, second projected levels of glucose in the patient over the second time frame; and outputting, by the one or more processors, the second projected levels of glucose for the second time frame.

In another example, the disclosure describes a non-transitory computer-readable storage medium having instructions stored thereon that, when executed, cause one or more processors to: determine an occurrence of a projection event that alters how first projected levels of glucose are to be output; automatically determine, based on the projection event, a second time frame that differs from a first time frame over which the first projected levels of glucose were projected; obtain a current glucose level of the patient; obtain, based on the current glucose level, second projected levels of glucose in the patient over the second time frame; and output the second projected levels of glucose for the second time frame,

The details of one or more aspects of the disclosure are set forth in the accompanying drawings and the description below, Other features, objects, and advantages of this disclosure will be apparent from the description and drawings, and from the claims.

### BRIEF DESCRIPTION OF DRAWINGS

FIG. 1 is a block diagram illustrating an example system for delivering or guiding therapy dosage, in accordance with one or more examples described in this disclosure.

FIG. 2 is a block diagram illustrating another example system for delivering or guiding therapy dosage, in accordance with one or more examples described in this disclosure.

FIG. 3 is a block diagram illustrating another example system for delivering or guiding therapy dosage, in accordance with one or more examples described in this disclosure..

FIGS. 4A and 4B are diagrams illustrating user interfaces of the patient device discussed with respect to the examples of FIGS. 1-3 in presenting a graphical alert according to various aspects of the techniques described in this disclosure.

FIGS. 5A-5C are diagrams illustrating user interfaces of the patient device discussed with respect to the examples of FIGS. 1-3 in presenting a graphical alert according to various aspects of the techniques described in this disclosure.

FIGS, 6A-6C are diagrams illustrating user interfaces of the patient device discussed with respect to the examples of FIGS. 1-3 in automatically switching between projection modes according to various aspects of the techniques described in this disclosure.

FIG. 7 is a block diagram illustrating an example of the patient device shown in FIGS. 1-3, in accordance with one or more examples described in this disclosure.

FIG, 8 is a flowchart illustrating example operation of the patient device shown in FIGS. 1-3 and 7 in performing various aspects of the automated alert disabling techniques.

FIG. 9 is a flowchart illustrating example operation of the patient device shown in FIGS. 1-3 and 7 in performing various aspects of the automated projection mode switching techniques.

### DETAILED DESCRIPTION

Various aspects of techniques for managing glucose level in a patient are described in this disclosure. Monitoring of glucose levels in patients may include an alert function in which a patient device may obtain data indicative of current glucose levels in the patient. The patient device and/or the backend servers may determine, based on the current glucose levels, projected glucose levels over a time frame (e.g., 1 hour, 2 hours, 4 hours, 8 hours, etc.). When the projected glucose levels outside of a prescribed range (which may be patient specific and set via the patient device, a physician's or other caregiver's programming, device, etc.), the patient device may present a graphical alert indicating that the projected glucose levels will leave the prescribed range.

The patient may review the graphical alert and dismiss the graphical alert when intending to inject insulin or consume carbohydrates (or, in other words, eat a meal or snack). While such intentions may be input into the patient device, the patient may become distracted or otherwise forget to enter the insulin or consume carbohydrates. As such, the patient device may continually present the graphical alerts (and possibly other audible or haptic alerts) in an effort to warn the patient of the hypoglycemic event or hyperglycemic event. Presentation of such alerts may result in wasted processor cycles, memory bandwidth, memory storage space, or other computing resources of the patient device when the patient intends to address the hypoglycemic event or hyper-glycemic event.

Furthermore, as the patient may forgo informing the patient device of various intentions, the patient device may present projected glucose levels that fail to take account of the patient intentions. For example, the patient may eat a meal, exercise, take a nap, become ill, or act in unexpected ways (e.g., eat abnormal amounts of food, such as during a holiday) that are not communicated to the patient device via entering accurately such events or intentions. The patient device may then present projected levels of glucose for time frames that do not apply to the current context in which the patient is acting. Inaccurately presenting projected levels of glucose may result in wasted processor cycles, memory bandwidth, memory storage space, or other computing resources of the patient device, as such projected levels of glucose do not enable the patient to address any hypoglycemic event or hyperglycemic event.

In accordance with various aspects of the techniques described in this disclosure, the patient device may automatically dismiss or otherwise disable alerts responsive to detecting a maintenance event that alters the projected levels of glucose such that the projected levels of glucose do not leave, i.e., go outside, the prescribed range. That is, the patient device may obtain an indication of various maintenance events, such as a meal event indicative of the patient eating a meal or an insulin injection event in which the patient receives insulin, and update or otherwise revise the projected levels of glucose to reflect the maintenance event. The patient device may then monitor the projected levels of glucose, but in the interim may disable the alert for a temporary period of time (determined based on the revised projected levels of glucose). After expiration of the temporary period of time, the patient device may reenabie the alert (e.g., output the alert again),

By dismissing or otherwise disabling the alert. for the temporary period of time, the patient device may avoid needlessly consuming processor cycles, memory bandwidth, memory storage space, or other computing resources, such as power, that would have otherwise been consumed by repeatedly presenting the alert as a result of not accounting for the maintenance event. Furthermore, by disabling the alert, the patient device may avoid annoying the patient with repeated alerts or desensitizing the patient to alerts, which may raise patient engagement with the patient device and thereby improve projections of glucose levels (as the patient may be more willing to enter information regarding insulin delivery, meal consumption, exercise, sleep, and the like).

Moreover, rather than present projected levels of glucose for time frames that are not relevant to the patient in the current context, various aspects of the techniques may enable the patient device to automatically determine projection events that result in dynamic selection of projection modes between the different time frames. For example, the patient device may determine a time of day event (e.g., a meal event, a sleep event, etc. that occurs at a given time of day), a physiological event (es., an illness event, a menstruation event, a medication event, etc.), a lifestyle event (e.g., a holiday event, a vacation event, an exercise event, etc.) and/or a data-driven event (e.g., a missing data event, a prediction inaccuracy event, a historical event, etc.). The patient device may then automatically determine, responsive to the projection event, a second time frame (e.g, with a different duration of time than the first time frame) for which to obtain projected levels of glucose. The patient device may present the revised projected levels of glucose for the determined time frame that better facilitates understanding of the projected levels of glucose in the current context

As such, by automatically selecting a time frame by which to project levels of glucose, the patient device may avoid needlessly consuming processor cycles, memory bandwidth, memory storage space, or other computing resources that would have otherwise been consumed by presenting projected levels of glucose that do not account for the current context (as indicated by the projection event). Again, by projecting the glucose levels for the time frame that better fits the current context in which the patient is acting, the patient device may avoid annoying the patient with what would appear to be inaccurate information, which may raise patient engagement with the patient device and thereby improve projections of glucose levels (as the patient may be more willing to enter information regarding insulin delivery, meal consumption, exercise, sleep, and the like).

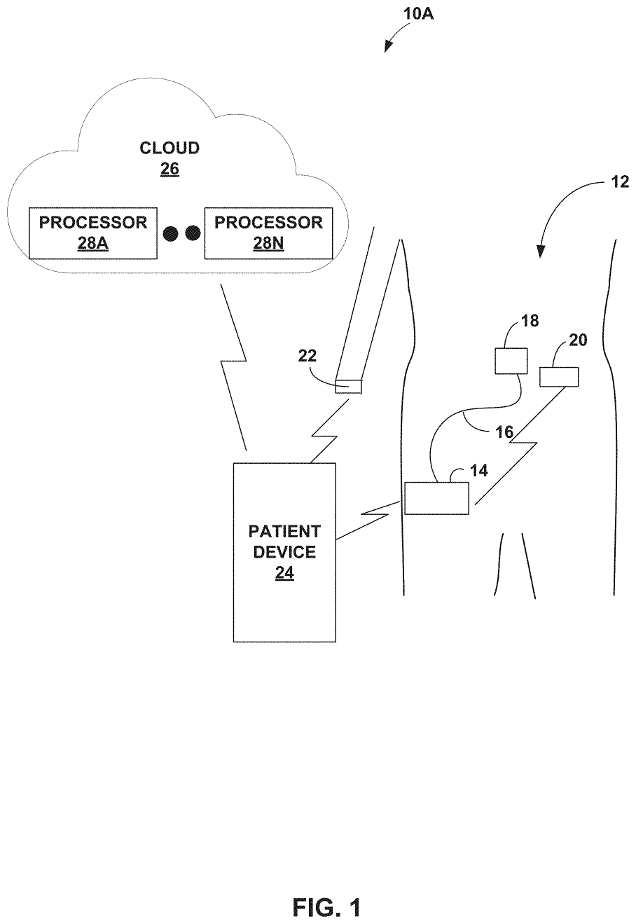

FIG. 1 is a block diagram illustrating an example system for delivering or guiding therapy dosage, in accordance with one or more examples described in this disclosure. FIG, 1 illustrates system 10A that includes a patient 12, an insulin pump 14, a tubing 16, an infusion set 18, a sensor 20 (e.g., glucose sensor), a wearable device 22, a patient device 24, and a cloud 26. Cloud 26 represents a local, wide area or global computing network including one or more processors 28A-28N (“one or more processors 28”). In some examples, system 10A may be referred to as continuous glucose monitoring (CGM) system or closed-loop system 10A.

Patient 12 may be diabetic (e.g., Type 1 diabetic or Type 2 diabetic), and therefore, the glucose level in patient 12 may be uncontrolled without delivery of supplemental insulin. For example, patient 12 may not produce sufficient insulin to control the glucose level or the amount of insulin that patient 12 produces may not be sufficient due to insulin resistance that patient 12 may have developed.

To receive the supplemental insulin, patient 12 may carry insulin pump 14 that couples to tubing 16 for delivery of insulin into patient 12. Infusion set 18 may connect to the skin of patient 12 and include a cannula to deliver insulin into patient 12. Sensor 20 may also be coupled to patient 12 to measure glucose level in patient 12. Insulin pump 14, tubing 16, infusion set 18, and sensor 20 may together form an insulin pump system. One example of the insulin pump system is the MINIMED™ 670G INSULIN PUMP SYSTEM by Medtronic, Inc. However, other examples of insulin pump systems may be used and the example techniques should not be considered limited to the MINIMED™ 670G INSULIN PUMP SYSTEM.

Insulin pump 14 may be a relatively small device that patient 12 can place in different locations. For instance, patient 12 may clip insulin pump 14 to the waistband of pants worn by patient 12. In some examples, to be discreet, patient 12 may place insulin pump 14 in a pocket. In general, insulin pump 14 can be worn in various places (or implanted within patient 12) and patient 12 may place insulin pump 14 in a location based on the particular clothes patient 12 is wearing.

To deliver insulin, insulin pump 14 includes one or more reservoirs (e.g., two reservoirs). A reservoir may be a plastic cartridge that holds up to N units of insulin (e.g., up to (K) units of insulin) arid is locked into insulin pump 14. Insulin pump 14 may be a battery powered device that is powered by replaceable batteries and/or rechargeable batteries.

Tubing 16. sometimes called a catheter, connects on a first end to a reservoir in insulin pump 14 and connects on a second end to infusion set 18. Tubing 16 may carry the insulin from the reservoir of insulin pump 14 to patient 12. Tubing 16 may be flexible, allowing for looping or bends to minimize concern of tubing 16 becoming detached from insulin pump 14 or infusion set 18 or concern from tubing 16 breaking.

Infusion set 18 may include a thin cannula that patient 12 inserts into a layer of fat under the skin (e.g., subcutaneous connection). Infusion set 18 may rest near the stomach of patient 12. The insulin travels from the reservoir of insulin pump 14, through tubing 16, and through the cannula in infusion set 18, and into patient 12. In some examples, patient 12 may utilize an infusion set insertion device. Patient 12 may place infusion set 18 into the infusion set insertion device, and with a push of a button on the infusion set insertion device, the infusion set insertion device may insert the cannula of infusion set 18 into the layer of fat of patient 12, and infusion set 18 may rest on top of the skin of the patient with the cannula inserted into the layer of fat of patient 12.

Sensor 20 may include a sensor that is inserted under the skin of patient 12, such as near the stomach of patient 12 or in the arm of patient 12 (e.g., subcutaneous connection). The sensor of sensor 20 may be configured. to measure the interstitial glucose level, which is the glucose found in the fluid between the cells of patient 12 (which also may be referred to as sensor glucose—SG—level that is distinguished from blood glucose—BG—level given that SG measures the glucose in the interstitial fluid between cells compared to the BG that measures glucose in the blood). Sensor 20 may be configured to continuously or periodically sample the glucose level and rate of change of the glucose level over time.

In one or more examples, insulin pump 14 and sensor 20 may together form a closed-loop therapy delivery system. For example, patient 12 may set a target glucose level, usually measured in units of milligrams per deciliter, on insulin pump 14. Insulin pump 14 may receive the current glucose level from sensor 20, and in response may increase or decrease the amount of insulin delivered to patient 12. For example, if the current glucose level is higher than the target glucose level, insulin pump 14 may increase the insulin. If the current glucose level is lower than the target glucose level, insulin pump 14 may temporarily cease or, in other words, refrain from delivery of the insulin. Insulin pump 14 may be considered as an example of an automated insulin delivery (AID) device. Other examples of AID devices may be possible, and the techniques described in this disclosure may be applicable to other AID devices.

For example, insulin pump 14 and sensor 20 may be configured to operate together to mimic some of the ways in which a healthy pancreas works. Insulin pump 14 may be configured to deliver basal insulin, which is a small amount of insulin released continuously throughout the day. There may be times when glucose levels increase, such as due to eating or some other activity that patient 12 undertakes such as sleep, exercise, etc. Insulin pump 14 may be configured to deliver bolus insulin on demand in association with food intake or to correct an undesirably high glucose level in the bloodstream. In one or more examples, if the glucose level rises above a target level, then insulin pump 14 may increase the bolus insulin to address the increase in. glucose level. Insulin pump 14 may be configured to compute basal and bolus insulin delivery, and deliver the basal and bolus insulin accordingly. For instance, insulin pump 14 may determine the amount of basal insulin to deliver continuously, and then determine the amount of bolus insulin to deliver to reduce glucose level in response to an increase in glucose level due to eating other ingestion of carbohydrates) or some other event.

Accordingly, in some examples, sensor 20 may sample glucose level and. rate of change in glucose level over time. Sensor 20 may output the glucose level to insulin pump 14 (e.g., through a wireless link connection, like Bluetooth™, BLE, WiFi®, or other personal area network protocol and/or wireless protocol). Insulin pump 14 may compare the glucose level to a target glucose range, or in other words, prescribed glucose range (e.g., as set by patient 12 or clinician), and adjust the insulin dosage based on the comparison.

As described above, patient 12 or a clinician may set the prescribed glucose range on insulin pump 14. There may be various ways in which patient 12 or the clinician may set the prescribed glucose range on insulin pump 14, As one example, patient 12 or the clinician may utilize patient device 24 to communicate with insulin pump 14. Examples of patient device 24 include mobile devices, such as smartphones or tablet computers, laptop computers, and the like. In examples, patient device 24 may be a special programmer or controller for insulin pump 14. Although FIG. 1 illustrates one patient device 24, in some examples, there may be a plurality of patient devices. For instance, system 10A may include a mobile device and a controller, each of which are examples of patient device 24. For ease of description only, the example techniques are described with respect to patient device 24, with the understanding that patient device 24 may be one or more patient devices.

Patient device 24 may also be configured to interface with sensor 20. As one example, patient device 24 may receive information (e.g,, glucose level or rate of change of glucose level) directly from sensor 20 (e.g., through the wireless link). As another example, patient device 24 may receive information from sensor 20 through insulin pump 14, where insulin pump 14 relays the information between patient device 24 and sensor 20.

In one or more examples, patient device 24 may display a user interface with which patient 12 or the clinician may control insulin pump 14. For example, patient device 24 may display a screen that allows patient 12 or the clinician to enter the prescribed glucose range. As another example, patient device 24 may display a screen that outputs the current glucose level. In some examples, patient device 24 may output notifications (or, in other words, alerts) to patient 12, such as notifications if the glucose level is too high or too low, as well as notifications regarding any action that patient 12 needs to take. For example, if the batteries of insulin pump 14 are low on charge, then insulin pump 14 may output a low battery indication to patient device 24, and patient device 24 may in turn output a notification to patient 12 to replace or recharge the batteries.

Controlling insulin pump 14 through patient device 24 is one example, and should not be considered limiting. For example, insulin pump 14 may include a user interface (e.g., pushbuttons) that allow patient 12 or the clinician to set the various prescribed glucose ranges of insulin pump 14. Also, in some examples, insulin pump 14 itself, or in addition to patient device 24, may be configured to output notifications to patient 12. For instance, if the glucose level is too high or too low, insulin pump 14 may output an audible or haptic output. As another example, if the battery is low, then insulin pump 14 may output a low battery indication on a display of insulin pump 14.

The above describes examples ways in which insulin pump 14 deliver insulin to patient 12 based on the current glucose levels (e.g., as measured by sensor 20). In Kale cases, there may be therapeutic gains by proactively delivering insulin to patient 12, rather than reacting to when glucose levels become too high or too low.

The glucose level in patient 12 may increase due to particular user actions. As one example, the glucose level in patient 12 may increase due to patient 12 engaging in an activity like eating or exercising. In some examples, there may be therapeutic gains if it is possible to determine that patient 12 is engaging in the activity, and delivering insulin based on the determination that patient 12 is engaging in the activity.

For example, patient 12 may forget to cause insulin pump 14 to deliver insulin after eating, resulting in an insulin shortfall. Alternatively, patient 12 may cause insulin pump 14 to deliver insulin after eating but may have forgotten that patient 12 previously caused insulin pump 14 to deliver insulin for the same meal event, resulting in an excessive insulin dosage. Also, in examples where sensor 20 is utilized, insulin pump 14 may not take any action until after the glucose level is greater than a target level (which is a high or low threshold in the prescribed glucose range). By proactively determining that patient 12 is engaging in an activity, insulin pump 14 may be able to deliver insulin in such a manner that the glucose level does not rise above the target level or rises only slightly above the target level (i.e., rises by less than what the glucose level would have risen if insulin were not delivered proactively). In some cases, by proactively determining that patient 12 is engaging in an activity and delivering insulin accordingly, the glucose level of patient 12 may increase more slowly.

Although the above describes proactive determination of patient 12 eating and delivering insulin accordingly, the example techniques are not so limited. The example techniques may be utilized for proactively determining an activity that patient 12 is undertaking (e.g., eating, exercising, sleeping, driving, etc.). Insulin pump 14 may then deliver insulin based on the determination. of the type of activity patient 12 is undertaking.

As illustrated, patient 12 may wear wearable device 22. Examples of wearable device 22 include but are not limited to a smartwatch or a fitness tracker, either of which may, in some examples, be configured to be worn on a patient's wrist or arm, e.g., as a wrist watch or band. In one or more examples, wearable device 22 includes one or more accelerometers (e.g., a six-axis accelerometer). Wearable device 22 may be configured to determine one or more movement characteristics of patient 12. Examples of the one or more movement characteristics include values relating to frequency, amplitude, trajectory, position, velocity, acceleration and/or pattern of movement currently or over time. The frequency of movement of the patient's arm may refer to how many times patient 12 repeated a movement within a certain time (e.g., such as frequency of movement back and forth between two positions).

Patient 12 may wear wearable device 22 on his or her wrist. However, the example techniques are not so limited. Patient 12 may wear wearable device 22 on a finger, forearm, or bicep. In general, patient 12 may wear wearable device 22 anywhere that can be used to determine movements indicative of eating, such as movement characteristics of the arm.

The manner in which patient 12 is moving his or her arm (i.e., the movement characteristics) may refer to the direction, angle, and orientation of the movement of the arm of patient 12, including values relating to frequency, amplitude, trajectory, position, velocity, acceleration and/or pattern of movement instantaneously or over time. As an example, if patient 12 is eating, then the arm of patient 12 will be oriented in a particular way (e.g., thumb is facing towards the body of patient 12), the angle of movement of the arm will be approximately a 90-degree movement (e.g., starting from plate to mouth), and the direction of movement of the arm. will be a path that follows from plate to mouth. The forward,-backward, up/down, pitch, roll, yaw measurements from wearable device 22 may be indicative of the manner in which patient 12 is moving his or her arm. Also, patient 12 may have a certain frequency at which patient 12 moves his or her arm or a pattern at which patient 12 moves his or her arm that is more indicative of eating, as compared to other activities, like smoking or vaping, where patient 12 may raise his or her arm to his or her mouth.

Although the above description describes wearable device 22 as being utilized to determine whether patient 12 is eating, wearable device 22 may be configured to detect movements of the arm of patient 12 (e.g., one or more movement characteristics), and the movement characteristics may be utilized to determine an activity undertaken by patient 12. For instance, the movement characteristics detected by wearable device 22 may indicate whether patient 12 is exercising, driving, sleeping, etc, As another example, wearable device 22 may indicate posture of patient 12, which may align with a posture for exercising, driving, sleeping, eating, etc. Another term for movement characteristics may be gesture movements. Accordingly, wearable device 22 may be configured to detect movements (e.g., movement characteristics of the arm of patient 12) and/or posture, where the movement and/or posture may be part of various activities (e.g., eating, exercising, driving, sleeping, etc.).

In some examples, wearable device 22 may be configured to determine, based on the detected movement (e.g., movement characteristics of the arm of patient 12) and/or posture, the particular activity patient 12 is undertaking. For example, wearable device 22 may be configured to determine whether patient 12 is eating, exercising, driving, sleeping, etc. In some examples, wearable device 22 may output information indicative of the movement of the arm of patient 12 and/or posture of patient 12 to patient device 24, and patient device 24 may be configured to determine the activity patient 12 is undertaking.

Wearable device 22 and/or patient device 24 may be programmed with information that wearable device 22 and/or patient device 24 utilize to determine the particular activity patient 12 is undertaking For example, patient 12 may undertake various activities throughout the day where the movement characteristics of the arm of patient 12 may be similar to the movement characteristics of the arm of patient 12 for a particular activity, but patient 12 is not undertaking that activity. As one example, patient 12 yawning and cupping his or her mouth may have a similar movement as patient 12 eating. Patient 12 picking up groceries may have similar movement as patient 12 exercising. Also, in some examples, patient 12 may be undertaking a particular activity, but wearable device 22 and/or patient device 24 may fail to determine that patient 12 is undertaking the particular activity.

Accordingly, in one or more examples, wearable device 22 and/or patient device 24 may “learn” to determine whether patient 12 is undertaking a particular activity. However, the computing resources of wearable device 22 and patient device 24 may be insufficient to perform the learning needed to determine whether patient 12 is undertaking a particular activity. It may be possible for the computing resources of wearable device 26 and patient device 24 to be sufficient to perform the learning, but for ease of description only, the following is described with respect to one or more processors 28 in cloud 26.

As illustrated in FIG. 1, system 10A includes cloud 26 that includes one or more processors 28. Cloud 26 represents a cloud infrastructure that supports one or more processors 28 on which applications or operations requested by one or more users run. For example, cloud 26 provides cloud computing for using one or more processors 28, to store, manage, and process data, rather than by personal device 24 or wearable device 22. One or more processors 28 may share data or resources for performing computations, and may be part of computing servers, web servers, database servers, and the like. One or more processors 28 may be in servers within a datacenter or may be distributed across multiple datacenters. In some cases, the datacenters may be in different geographical locations.

One or more processors 28, as well as other processing circuitry described herein, can include any one or more microprocessors, digital signal processors (DSPs), application specific integrated circuits (ASICs), field programmable gate arrays (FPGAs), or any other equivalent integrated or discrete logic circuitry, as well as any combinations of such components. The functions attributed one or more processors 28, as well as other processing circuitry described herein, herein may be embodied as hardware, firmware, software or any combination thereof.

One or more processors 28 may be implemented as fixed-function circuits, programmable circuits, or a combination thereof. Fixed-function circuits refer to circuits that provide particular functionality, and are preset on the operations that can be performed. Programmable circuits refer to circuits that can be programmed to perform various tasks, and provide flexible functionality in the operations that can be performed. For instance, programmable circuits may execute software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. In some examples, the one or more of the units may be distinct circuit blocks (fixed-function or programmable), and in some examples, the one or more units may be integrated circuits. One or more processors 28 may include arithmetic logic units (ALUs), elementary function units (EFUs), digital circuits, analog circuits, and/or programmable cores, formed from programmable circuits. In examples where the operations of one or more processors 28 are performed using software executed by the programmable circuits, memory (e.g., on the servers) accessible by one or more processors 28 may store the object code of the software that one or more processors 28 receive and execute.

In some examples, one or more processors 28 may be configured to determine patterns from the indications of movements (e.g., as one or more movement characteristics determined by wearable device 22), and configured to determine a particular activity patient 12 is undertaking. One or more processors 28 may provide responsive real-time cloud services that can, on a responsive real-time basis, determine the activity patient 12 is undertaking, and in some examples, provide recommended therapy insulin dosage amount). Cloud 26 and patient device 24 may communicate via through a carrier network, such as a cellular network, or via any other standard communication network.

For example, as described above, in some examples, wearable device 22 and/or patient device 24 may be configured to determine that patient 12 is undertaking an activity or other event. However, in some examples, patient device 24 may output information indicative of the movement of the arm of patient 12 to cloud 26, and possibly with other contextual information like location or time of day. One or more processors 28 of cloud 26 may then determine the activity (or, in other words, event) patient 12 is undertaking. Insulin pump 14 may then deliver insulin based on the determined activity of patient 12.

One example way in which one or more processors 28 may be configured to determine that patient 12 is undertaking an activity and determine therapy to deliver is described in U.S. Patent Publication No. 2020/0135320

A1. In general, one or more processors 28 may first go through an initial “learning” phase, in which one or more processors 28 receive information to determine behavior patterns of patient 12 via. indications of movements specific to patient 12. Some of this information may be provided by patient 12. For example, patient 12 may be prompted or may himself/herself enter information into patient device 24 indicating that patient 12 is undertaking a particular activity, the length of the activity, and other such information that one or more processors 28 can utilize to predict behavior of patient 12. After the initial learning phase, one or more processors 28 may still update the behavior patterns based on more recent received information, but require fewer to no information from patient 12.

In the initial learning phase, patient 12 may provide information about the dominant hand of patient 12 (e.g., right or left-handed) and where patient 12 wears wearable device 22 (e.g., around the wrist of right hand or left hand). Patient 12 may be instructed to wear wearable device 22 on the wrist of the hand patient 12 uses to eat. Patient 12. may also provide information about the orientation of wearable device 22 (e.g., face of wearable device 22 is on the top of the wrist or bottom of the wrist).

In the initial learning phase, patient 12 may enter, proactively or in response to prompt/query, information through patient device 24) indicating that patient 12 is engaging in an activity or, again in other words, event. During this time, wearable device 22 may continuously determine the one or more movement characteristics (e.g., gesture) and/or posture of patient 12, and output such information to patient device 24 that relays the information to processors 28. Processors 28 may store information of the one or more movement characteristics of movement of the arm of patient 12 during the activity to later determine whether patient 12 is engaging in that activity (e.g., whether the received information of the manner and frequency of movement of the arm of patient 12 aligns with the stored information of the manner and frequency of movement of the arm of patient 12 when patient 12 was known to be engaging in that activity).

The above describes arm movement as a factor in determining whether patient 12 is engaging in the identified event. However, there may be various other factors that can be used separately or in combination with arm movement to determine whether patient 12 is engaging in the event. As one example, patient 12 may engage in the event at regular time, intervals. As another example, patient 12 may engage in the event at certain locations. In the initial learning phase, when patient 12 enters that be or she is engaging in the event (e.g., through patient device 24), patient device 24 may output information about the time of day and the location of patient 12. For example, patient device 24 may be equipped with a positioning device, such as global positioning system (GPS) unit, and patient device 24 may output location information determined by the GPS unit. There may be other ways to determine location such as based on Wi-Fi connection and/or access to 4G/5G LTE cellular connections, or some other form of access, such as based on telecom database tracking device location of patient device 24. Time of day and location are two examples of contextual information that can be used to determine whether patient 12 is engaging in the activity.

However, there may be other examples of contextual information for patient 12 such as sleep pattern, body temperature, stress level (e.g., based on pulse and respiration), heart rate, etc, In general, there may be various biometric sensors to measure temperature, pulse/heart rate, breathing rate, etc.), which may be part of wearable device 22 or may be separate sensors. In some examples, the biometric sensors may be part of sensor 20.

The contextual information for patient 12 may include conditional information. For example, patient 12 may eat every 3 hours, but the exact times of when patient 12 eats may be different. In some examples, the conditional information may be a determination of whether patient 12 has eaten and if a certain amount of time (e.g., 3 hours) has passed since patient 12 ate. In general, any information that establishes a pattern of behavior may be utilized to determine whether patient 12 is engaging in a particular activity.

Processors 28 may utilize artificial intelligence, such as machine-learning or other data analytics techniques, based on the information determined by and/or collected by wearable device 22 and patient device 24 to determine whether patient 12 is engaging in the activity. As one example, during the initial learning phase, one or more processors 28 may utilize neural network techniques. For example, one or more processors 28 may receive training data from patient 12 that is used to train a classifier module executing on processors 28. As described above, processors 28 may receive the training data based on patient confirmation when patient device 24 and/or wearable device 22 determine, based on manner and frequency of movement of the arm of patient 12, that patient 12 is engaging in the activity (e.g., a gesture that aligns with movement of arm for eating). Processors 28 may generate and store a labeled data record that includes the features related to the movement, along with other contextual features, such as time of day or location. Processors 28 may train the classifier on a labeled dataset that includes multiple labeled data records, and processors 28 may use the trained classifier model to more accurately detect the start of a food intake event.

Other examples that may be used for neural networks include behavior patterns. For example, patient 12 may only eat a particular food after exercising, and always eats that particular food after exercising. Patient 12 may eat, or in other words, consume a meal or snack, at a particular time and/or place. Although described with respect to eating, there may be various conditions that together indicate a pattern in behavior of patient 12 for different activities.

As another example, one or more processors 28 may utilize k-means clustering techniques to deter mine whether patient 12 is engaging in the event. For example, during the initial learning phase one or more processors 28 may receive different types of contextual information and form clusters, where each cluster represents a behavior of patient 12 (e.g., eating, sleeping, exercising, etc.). For example, patient 12 may enter information (e.g., into patient device 24) indicating that he or she is exercising, e.g.. walking, running, etc. Processors 28 may utilize all the contextual information received when patient 12 is exercising to form a first cluster associated with exercising. Patient 12 may enter information (e.g., into patient device 24) indicating that be or she is eating. Processors 28 may utilize all the contextual information received when patient 12 is eating to form a second cluster associated with eating, and so on. Then, based on received contextual information, processors 28 may determine which cluster aligns with the contextual information, and determine the event patient 12 is undertaking, As described in more detail, the type of event, and a prediction of when the event will occur, may be utilized to determine when to delivery insulin therapy. There may be other examples of machine learning, and the example techniques are not limited to any particular machine learning technique.

There may be various other ways in which processors 28 may determine the event patient 12 is undertaking. This disclosure provides some example techniques for determining the event patient 12 is undertaking, but the example techniques should not be considered limiting.

During the initial learning phase, patient 12 may also enter information about the event that patient 12 is undertaking. For example, with eating, patient 12 may enter information indicating what patient 12 is eating and/or how many carbohydrates there are in the food that patient 12 is eating. As one example, at 9:00 every morning, patient 12 may enter that be or she is having a bagel or enter that the patient 12 is consuming 48 grams of carbohydrates.

In some examples, processors 28 may be configured to determine an amount of insulin (e.g., therapy dosage of bolus insulin) to deliver to patient 12. As one example, memory accessible by one or more processors 28 may store patient parameters of patient 12 (e.g., weight, height, etc.). The memory may also store a look-up table that indicates an amount of bolus insulin that is to be delivered for different patient parameters and different types of foods. Processors 28 may access the memory and, based on the type of food patient 12 is eating and patient parameters (each of which influence projected levels of glucose that have been identified specially for patient 12), determine the amount of bolus insulin that patient 12 is to receive.

As another example, processors 28 may be configured to utilize a “digital twin” of patient 12 to determine an amount of bolus insulin that. patient 12 is to receive. A digital twin may be a digital replica or model of patient 12. The digital twin may be software executing on processors 28 and/or patient device 24. The digital twin may receive, as input, information about what patient 12 is eating, Because the digital twin. is a digital replica of patient 12, the output from the digital twin may be information about what the glucose level of patient 12 may be after eating, as well as a recommendation of how much bolus insulin to deliver to patient 12 to control the increase the glucose level. Accordingly, a digital twin of patient 12 allows analysis of real-time data (e.g., what patient 12 is eating), impact on patient 12 (e.g., how much change there will be in the glucose level), and/or therapy recommendations (e.g., how much insulin to provide).

Accordingly, in one or more examples, processors 28 may utilize information about the movement characteristics of movement of arm, eating pace, quantity of food consumption, food content, etc., while also tracking other contextual information. Examples of the contextual information include location information, time of day, wake up time, amount of time since last eaten, calendar event, information about person patient 12 may be meeting, etc. Processors 28 may identify patterns and correlations between all these various factors to determine an activity patient 12 undertaking, like eating, walking, sleeping, driving, etc.

After the initial learning phase, processors 28 may automatically, (e.g. meaning with minimal or no input from patient 12), determine that patient 12 is undertaking a particular event, and determine an amount of bolus insulin to deliver based on the determination of the particular event. Processors 28 may output the recommendation of the amount of bolus insulin to deliver to patient device 24. Patient device 24, may then in turn, control insulin pump 14 to deliver the determined amount of insulin. As one example, patient device 24 may output to insulin pump 14 the amount of bolus insulin to deliver. As another example, patient device 24 may output a target glucose level, and insulin pump 14 may deliver the insulin to achieve the target glucose level. In some examples, processors 28 may output to patient device 24 information indicative of the target glucose level, and patient device 24 may output that information to insulin pump 16. All of these examples may be considered as examples of one or more processors 28 determining an amount of insulin to deliver to patient 12.

The above describes example ways in which to determine if patient 12 is undertaking an activity, determining an amount of insulin to deliver, and/or causing the amount of insulin to be delivered. The example techniques may require little to no intervention from patient 12. In this manner, there is an increase in likelihood that patient 12 will receive the correct amount of dosage of insulin at the right time, and. decrease in likelihood of human error causing issues (e.g., patient 12 forgetting to log meals, forgetting to take insulin, or taking insulin but forgetting to have taken insulin).

While the above example techniques may be beneficial in patient 12 receiving insulin at the right time, this disclosure describes example techniques to further proactively control delivery of insulin to patient 12 and/or otherwise facilitate therapy delivery in a manner that reduces needless operation of patient device 24 while improving engagement of patient 12 with patient device 24. As described above, monitoring of glucose levels in patient 12 may include an alert function in which patient device 24 may interface with backend servers (as represented by processors 28 of cloud 26) to obtain data indicative of current glucose levels in patient 12. Patient device 24 and/or the backend servers 28 (which is another way to refer to processors 28) may determine, based on the current glucose levels, projected glucose levels over a time frame (e.g., 1 hour, 2 hours, 4 hours, 8 hours, etc.). When the projected glucose levels exceed a prescribed range (e.g., above an upper threshold or below a lower threshold, either of which may be specific to patient 12 and set via patient device 24, a doctor's programming device, etc.), patient device 24 may present a graphical alert (or, in other words, a visual alert, which may include any combination of text, icons, images, graphics, etc.) indicating that the projected glucose levels will leave the prescribed range, i.e., drop below a lower bound of the range or rise above an upper bound of the range (and thereby enter a hyperglycemic event or hypoglycemic event).

Patient 12 may review the graphical alert and dismiss the graphical alert when intending to inject insulin or consume carbohydrates (or, in other words, eat a meal or snack). While such intentions may be input into patient device 24, patient 12 may become distracted or otherwise forget to enter the insulin or consume carbohydrates, As such, patient device 24 may continually present the graphical alerts (and possibly other audible or haptic alerts) in an effort to warn the patient of the upcoming hypoglycemic event or hyperglycemic event. Presentation of such alerts may result in wasted processor cycles, memory bandwidth, memory storage space, or other computing resources of the patient device when the patient intends to address the hypoglycemic event or hyperglycemic event.

Furthermore, as patient 12 may forgo informing the patient device of various intentions, patient device 24 may present projected glucose levels that fail to take account of the patient intentions. For example, patient 12 may eat a meal, exercise, take a nap, become ill, or act in unexpected ways (e.g., eat abnormal amounts of food, such as during a holiday or vacation) that are not communicated to patient device 24 via entering accurately such events or intentions. Patient device 24 may then present projected levels of glucose for time frames that do not apply to the current context in which patient 12 is acting. Inaccurately presenting projected levels of glucose may result in wasted processor cycles, memory bandwidth, memory storage space, or other computing resources of patient device 24, as such projected levels of glucose do not enable patient 12 to address any hypoglycemic event or hyperglycemic event.

In accordance with various aspects of the techniques described in this disclosure, patient device 24 may automatically dismiss or otherwise disable alerts responsive to detecting a maintenance event that alters the projected levels of glucose such that the projected levels of glucose do no leave the prescribed range (e.g., eschew from generating and/or outputting the alert). That is, patient device 24 may obtain an indication of a maintenance event, such as a meal event indicative of patient 12 eating a meal or an insulin injection event in which patient 12 receives insulin, and update or otherwise revise the projected levels of glucose to reflect the maintenance event. Patient device 24 may then monitor the projected levels of glucose, but in the interim may disable the alert for a temporary period of time (determined based on the revised projected levels of glucose). Patient device 24 may, for example, set the temporary period of time at 15 minutes to start with and may, depending on the context, set it up to 60 minutes.

By dismissing or otherwise disabling the alert for the temporary period of time, patient device 24 may avoid needlessly consuming processor cycles, memory bandwidth, memory storage space, or other computing resources that would have otherwise been consumed by repeatedly presenting the alert as a result of not accounting for the maintenance event. Furthermore, by disabling the alert, patient device 24 may avoid annoying the patient with repeated alerts, which may raise patient engagement with patient device 24 and thereby improve projections of glucose levels (as the patient may be more willing to enter information regarding insulin delivery, meal consumption, exercise, sleep, and the like).

Moreover, rather than present projected levels of glucose for time frames that are not relevant to patient 12 in the current context, various aspects of the techniques may enable patient device 24 to automatically determine projection events that result in dynamic selection of projection modes between the different time frames. For example, patient device 24 may determine a time of day event (e.g., a meal event, a sleep event, etc. that occurs at a given time of day), a physiological event (e.g., an illness event, a menstruation event, a medication event, etc.), a lifestyle event (e.g., a holiday event, a vacation event, an exercise event, etc.) and/or a data-driven event (e.g., a missing data event, a prediction inaccuracy event, a historical event, etc.). Patient device 24 may then automatically determine, responsive to the projection event, a second time frame for which to obtain projected levels of glucose. Patient device 24 may present the revised projected levels of glucose for the determined time frame that better facilitates understanding of the projected levels of glucose in the current context.

As such, by automatically selecting a time frame by which to projected levels of glucose, patient device 24 may avoid needlessly consuming processor cycles, memory bandwidth, memory storage space, or other computing resources that would have otherwise been consumed by presenting projected levels of glucose that do not account for the current context (as indicated by the projection event). Again, by projecting the glucose levels for the time frame that better addresses the current context in which patient 12 is acting, patient device 24 may avoid annoying patient 12 with what would appear to be inaccurate information, which may raise patient engagement with patient device 24 and thereby improve projections of glucose levels (as patient 12 may be more willing to enter information regarding insulin delivery, meal consumption, exercise, sleep, and the like).

In operation, patient device 24 may obtain projected levels of glucose in patient 12 over a time frame (e.g., one hour, two hours, four hours, eight hours, etc.). Although described with respect to two hour, four hour, and eight hour time frames, the techniques may be performed with respect to any time frame, such as between one to three hours, between three to six hours, and greater than six hours, or any other time frame over which projected glucose levels can be reasonably projected. Patient device 24 may employ standard physiological models that are adapted to be specific to patient 12 in order to, in some instances, project levels of glucose in patient 12 over the given time frame. In some examples, patient device 24 may obtain, from sensor 20, a current glucose level and then determine, based on the current glucose level, the projected levels of glucose in patient 12 over the time frame.

Patient device 24 may next determine whether the projected levels of glucose leave, i.e., are outside, a prescribed range. Such a prescribed range may be set by patient 12, a doctor or other health care provider. The prescribed range may have an upper threshold that identifies when patient 12 enters a hyperglycemic state (which may he referred to as a hyperglycemic event) and a lower range at which point patient 12 enters a hypoglycemic event (which may be referred to as a hypoglycemic event).

When the projected levels of glucose in patient 12 leave the prescribed range (thereby indicating that patient 12 may experience a hyperglycemic event when projected glucose levels exceed the upper threshold or a hypoglycemic event when projected glucose levels are below the lower threshold), patient device 24 may generate a graphical alert, based on alert data, that indicates that the projected levels of glucose will leave the prescribed range, drop to be less than a lower bound of the range or rise to be greater than a higher bound of the range. Such an alert may include an interface (e.g., a virtual button on a touchscreen) by which patient 12 may dismiss or, in other words, disable the graphical alert for a temporary period of time (which may be referred to as “snoozing” the graphical alert). While discussed as providing a virtual button by which dismiss the graphical alert, patient 12 may issue a voice command or make a gesture to dismiss the graphical alert.

Patient device 24 may adaptively determine the temporary period of time. That is, patient device 24 may determine a number of factors that influence the duration of the temporary period of time for which patient 12 may disable the graphical alert. For example, patient device 24 may determine an amount of time remaining until patient 12 may experience the next projected hyperglycemic event or hypoglycemic event, and set the temporary period of time based on this amount of time remaining until patient 12 may experience the next projected hyperglycemic event or hypoglycemic event.

However, as noted above, patient 12 may not respond to the graphical alert due to distractions or other contexts (e.g., at a party, attending a gathering, exercising, eating, sleeping, etc.). In some instances, patient 12 may perform some form of maintenance event, such as eating a meal to correct for a potential hypoglycemic event, or inject or otherwise receive insulin to correct for a potential hyperglycemic event. Patient 12 may, however, inadvertently forget to interface with patient device 24 to enter, or in other words, log, the maintenance event. Rather than continually reissue the graphical alert (which may occur periodically or based on some other context, such as location), patient device 24 may automatically detect the maintenance event that alters the projected levels of glucose such that the projected levels of glucose do not leave the prescribed range (e.g., exceed the upper threshold or go below the lower threshold).

In some examples, patient device 24 may interface with wearable device 22 to automatically detect a meal event indicating that patient 12 is currently eating a meal (where wearable device 22 determines indication of movements indicative of eating a meal or snack). Responsive to detecting the meal event, patient device 24 may interface with sensor 20 to determine a current level of glucose, and then obtain, based on the current level of glucose, a revised version of the projected levels of glucose. Patient device 24 may determine that the revised version of the projected levels of glucose do not leave the prescribed range.

In another example, patient device 24 may automatically detect delivery of insulin, or in other words, an insulin delivery event indicating that patient 12 has injected insulin. Based on the insulin injection event, patient device 24 may obtain the revised version of the projected levels of glucose, and determine that the revised version of the projected levels of glucose do not leave the prescribed range over the given time frame. In both examples, patient device 24 may automatically disable the graphical alert for the temporary period of time.

In addition, patient device 24 may automatically switch between different projection modes (e.g., switching between different time frames over which to project the levels of glucose). Patient device 24 may originally project glucose levels over a first time frame (such as over 2 hours). Patient device 24 may determine an occurrence of a projection event that alters how the projected levels of glucose is to be output. As noted above, patient device 24 may determine a time of day event (e.g., a meal event, a sleep event, etc. that typically occurs at a given time of day and/or place), a physiological event (e.g., an illness event, a menstruation event, a medication event, etc.), a lifestyle event (e.g., a holiday event, a vacation event, an exercise event, etc.) and/or a data-driven event (e.g., a missing data event, a prediction inaccuracy event, a historical event, etc.).

To illustrate, assume that patient 12 regularly eats dinner around 6 PM. Patient device 24 may train a model that operates on the time of day event at 6 PM in which patient 12 regularly consumes about 60 grams of carbohydrates during dinner at 6 PM. Patient device 24 may detect such time of day event and confirm such time of day event by way of a data driven event in which patient device 24 interfaces with wearable device 22 to automatically detect a data driven meal event by was of movements detected by wearable device 22, Responsive to detecting such a time of day event (and/or a data driven event), patient device 24 may automatically determine a second time frame that differs from the first time frame (e.g., four hours versus 2 hours).

Patient device 24 may then interface with sensor 20 to obtain a current glucose level of patient 12, and obtain, based on the current glucose level, projected second levels of glucose in patient 12 over the second time frame. Patient device 24 may then output the second projected levels of glucose for the second time frame, which may better allow patient 12 to assess the impact of the carbohydrates consumed during dinner.

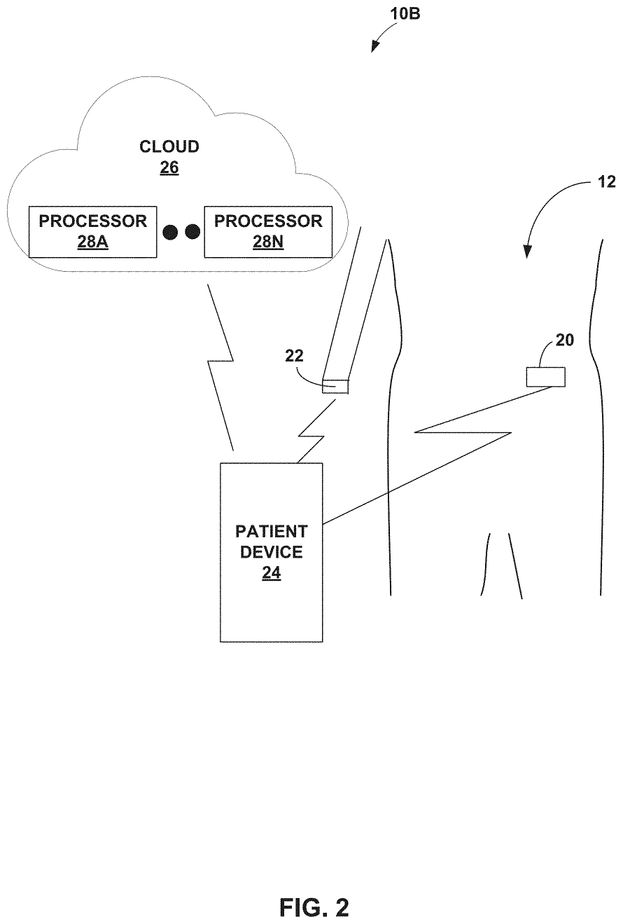

FIG. 2 is a block diagram illustrating another example system for delivering or guiding therapy dosage, in accordance with one or more examples described in this disclosure. FIG. 2 illustrates system 10B that is similar to system 10A of FIG. 1. However, in system 10B, patient 12 may not have insulin pump 14. Rather, patient 12 may utilize a manual injection device (e.g., a syringe) to deliver insulin. For example, rather than insulin pump 14 automatically delivering insulin, patient 12 (or possible a caretaker of patient 12) fill a syringe with insulin and inject himself or herself. In some examples, system 10B may be referred to as continuous glucose monitoring (CGM) system

Patient device 24 may interface, in this instance, with sensor 20 to determine a current glucose level (which may also be referred to a current level of glucose) and project, based on the current level of glucose, the levels of glucose over a given time frame (e.g., 2 hours). Patient device 24 may interface with wearable device 22 to detect the maintenance event (e.g., eating a meal or snack, which may refer to a smaller meal). Patient device 24 may also interface with sensor 20 to receive indications of the insulin delivery event, automatically detecting the manual injection of insulin. In this respect, patient device 24 may automatically detect the insulin delivery event through gestures, manual delivery via a syringe (via the glucose or other sensor), data from a smart pen or smart cap, and the like.

Likewise, patient device 24 may automatically detect projection events that enable switching of the projection modes. For example, patient device 24 may interface with wearable device 24 to determine that patient 12 is sleeping (based on movements) or exercising (based on movements, heart rate, GPS data, etc.). Patient device 24 may automatically switch, based on detection of the projection event, between different time frames over which to project the levels of glucose expected to occur in patient 12. To illustrate, patient device 24 may detect a time of day event in that patient 12 normally goes to sleep at 11 PM. At 10 PM, patient device 24 may automatically detect the time of day sleeping event, and automatically switch the time frame from four hours to eight hours without additional user input), projecting the levels of glucose over the next eight hours so that patient 12 may understand how the projected levels of glucose will occur while patient 12 is sleeping.

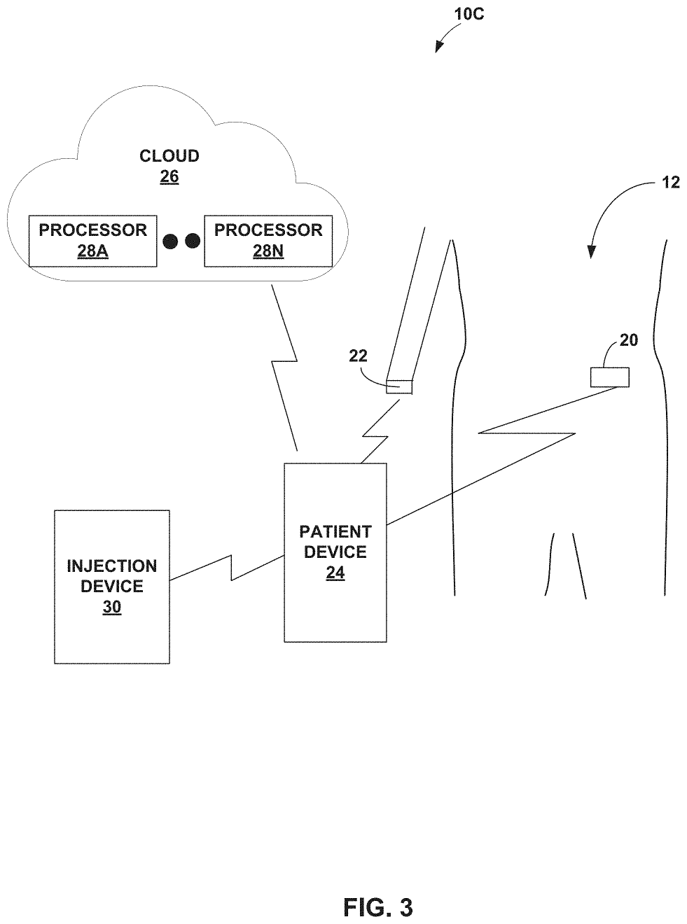

FIG. 3 is a block diagram illustrating another example system for delivering or guiding therapy dosage, in accordance with one or more examples described in this disclosure. FIG. 3 illustrates system IOC that is similar to system 10A of FIG. 1 and system 10B of FIG. 2. In system 10C, patient 12 may not have insulin pump 14. Rather, patient 12 may utilize injection device 30 to deliver insulin. For example, rather than insulin pump 14 automatically delivering insulin, patient 12 (or possible a caretaker of patient 12) may utilize injection device 30 to inject himself or herself.

Injection device 30 may be different than a syringe because injection device 30 may be a device that can communicate with patient device 24 and/or other devices in system IOC. Also, injection device 30 may include a reservoir, and based on information indicative of how much therapy dosage to deliver may be able to dose out that much insulin for delivery. In some examples, injection device 30 may be similar to insulin pump 14, but not worn by patient 12. One example of injection device 30 is an insulin pen or a smart insulin pen. Another example of injection device 30 may be an insulin pen with a smart cap, where the smart cap can be used to set particular doses of insulin.

The above examples described insulin pump 14, a syringe, and injection device 30 as example ways in which to deliver insulin. In this disclosure, the term “insulin delivery device” may generally refer to a device used to deliver insulin. Examples of insulin delivery device include insulin pump 14, a syringe, and injection device 30. As described, the syringe may be a device used to inject insulin but is not necessarily capable of communicating or dosing a particular amount of insulin. Injection device 30, however, may be a device used to inject insulin that may be capable of communicating with other devices or may be capable of dosing a particular amount of insulin. Injection device 30 may be powered (e.g., battery powered) device, and the syringe may be device that requires no power.

In this instance in which injection device 30 is powered, patient device 24 may interface with injection device 30, e.g., by wireless communication, to identify the insulin injection event as either a maintenance event or a projection event. As such, patient device 24 may automatically disable the graphical alert responsive to detecting the insulin delivery event for the temporary period of time. Moreover, patient device 24 may switch between projection modes (e.g., time frames over which to project levels of glucose) responsive to detecting the insulin injection event.

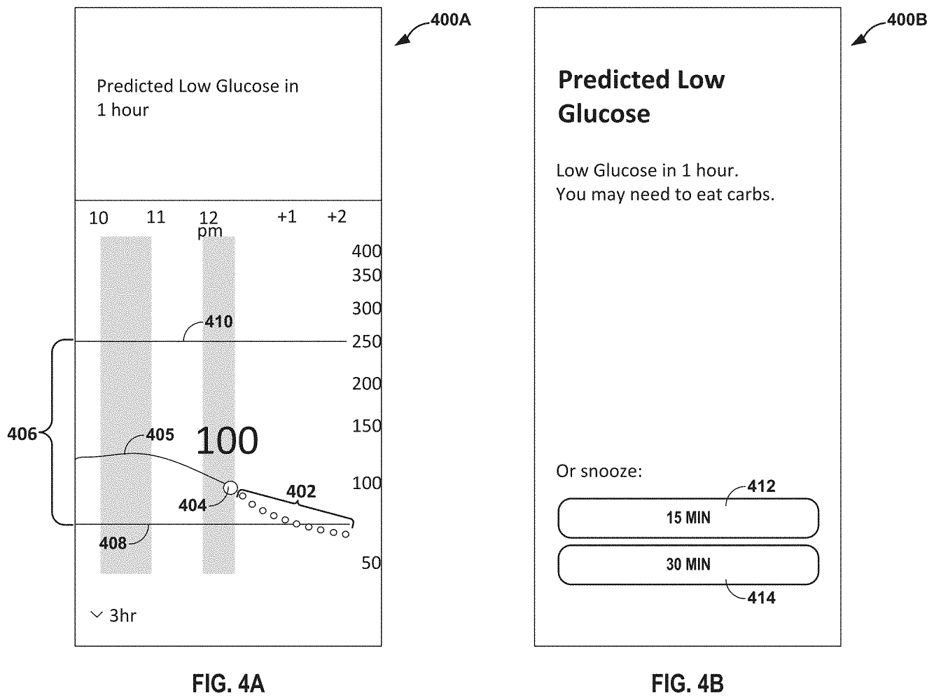

FIGS. 4A and 4B are diagrams illustrating user interfaces of the patient device discussed with respect to the examples of FIGS. 1-3 in presenting a graphical alert according to various aspects of the techniques described in this disclosure. In the example of FIG. 4A, patient device 24 may present a user interface 400A in which projected levels of glucose 402 are shown in graph form. Patient device 24 may determine projected levels of glucose 402 based on current glucose level 404 (of “100” as shown in the middle of the graph), which patient device 24 may obtain from sensor 20. The graph also shows past glucose measurements as a line 405 (starting from approximately 9:30 AM through the current time of approximately 12:15 PM).

The graph shows projected levels of glucose 402 over a time frame of two hours (shown as “+2” at the top of the graph) along with a prescribed range 406 having a lower threshold 408 and an upper threshold 410. At approximately one hour (shown as “+1”) from the current time, projected levels of glucose 402 are shown crossing below lower threshold 408. As such, patient device 24 provides, above the graph, the graphical alert indicating that “Predicted Low Glucose in 1 hour” to warn patient 12 of the possible hypoglycemic event.

In this respect, patient device 24 may generate the graphical alert based on alert data, which may include any aspect of the graphical alert shown in the example of FIG. 4A or 4B. The alert data may include an alert template that identifies where in graphical alert each aspect of the underlying data (e.g., projected levels of glucose 402, prescribed range 406, etc.) are to be aligned in the user interface relative to one another. The alert data may, in other words, identify formatting of the underlying data as well as positional information, background color, foreground color, text font, text color, or any other aspect of the user interface that facilitates presentation of the graphical alert.

In the example of FIG. 4B, patient device 24 may transition to the user interface shown in a user interface 400B in which another graphical alert is provided along with an option to “snooze” the graphical alert for either 15 minutes (“15 MIN”) or 30 minutes (“30 MIN”). Patient device 24 may construct each of the graphical alerts via. an alert template in which different text or images may be inserted into various portions of the alert template to form graphical alerts that warn patient 12 of different events. In the example of FIG. 4B, patient device 24 may populate the graphical alert with a title “Predicted Low Glucose” along with an indication of when the hypoglycemic event may occur (e.g., “Low Glucose in 1 hour.”) and an indication of a remedy to the hypoglycemic event (e.g., “You may need to eat carbs.”). The indication of when the hypoglycemic event may occur may be updated over time and presented on patient device 24 (e.g., “Low Glucose in 1 hour,” “Low Glucose in 45 minutes,” “Low Glucose in 30 minutes,” “Low Glucose in 5 minutes”).

The graphical alert shown in user interface 400B also includes the above noted snooze options that disable the graphical alert from occurring for either 15 minutes or 30 minutes. The graphical alert may include two interface elements 412 and 414 by which to prompt patient 12 to disable or otherwise “snooze” the graphical alert for the respective 15 minutes or 30 minutes, for example. Patient device 24 may select the temporary period of time by which to disable the alert based on a duration until the hypoglycemic event occurs (e.g., from the current time until the projected levels of glucose 402 cross below lower threshold 408, which occurs in one hour from the current time in the example of FIGS. 4A and 4B). Patient device 24 may, in this example, limit snooze durations to the one hour duration or some percentage of the duration (e.g, 75% and 50% of the duration in the example of FIG. 4B).

Furthermore, patient device 24 may automatically disable the graphical alert responsive to detecting the maintenance event, such as consumption of a meal as detected by way of movements provided by wearable device 22, through entry of a meal by patient 12 into patient device 24, etc. Patient device 24 may determine a revised version of projected levels of glucose 402 and compare the revised version of the projected levels of glucose to lower threshold 408 and upper threshold 410 to determine whether the revised version of projected levels of glucose 402 leave prescribed range 406, which is bounded by lower threshold 408 and upper threshold 410.

In some instances, patient device 24 may detect initiation of the maintenance event and next determine an amount associated with the maintenance event (e.g., a number of grams of carbohydrates consumed by patient 12 or units of insulin delivered to patient 12). Patient device 24 may determine, based on the amount associated with the maintenance event, that projected levels of glucose will still leave prescribed range 406. When presenting the graphical alert, patient device 24 may also present an audible alert, which can become disruptive (or, possibly annoying especially when performing the maintenance event). Responsive to determining that the amount is insufficient to prevent projected levels of glucose 402 from leaving prescribed range 406, patient device 24 may silence the audible alert, replacing the audible alert with a haptic alert (such that the audible alert is automatically disabled for the temporary period of time).

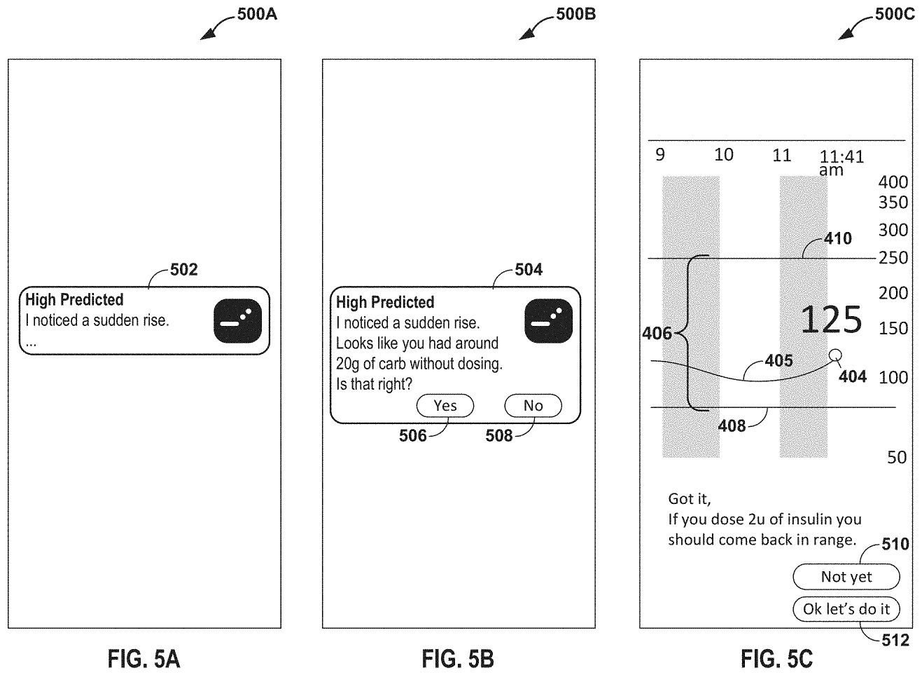

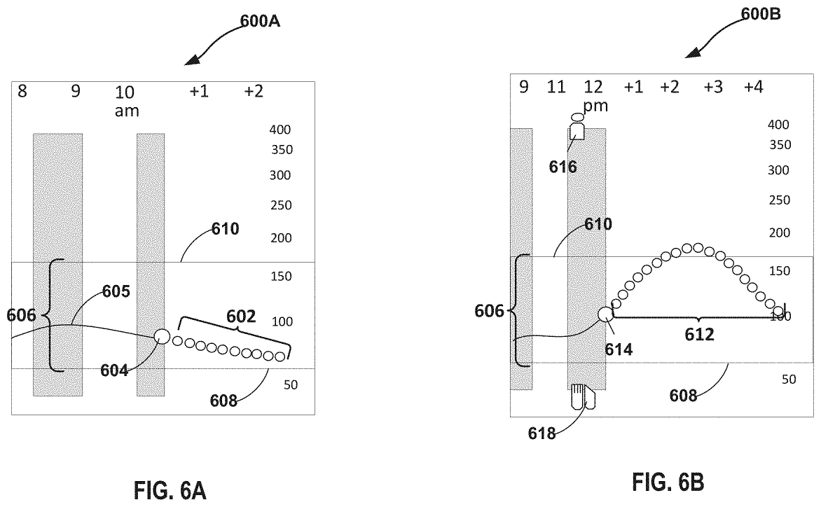

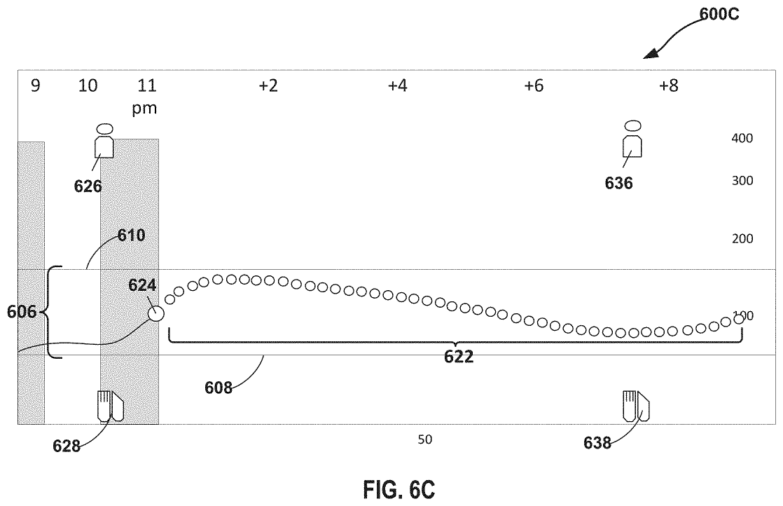

FIGS. 5A-5C are diagrams illustrating user interfaces of the patient device discussed with respect to the examples of FIGS. 1-3 in presenting a graphical alert according to various aspects of the techniques described in this disclosure. In the example of FIG. 5A, patient device 24 may initially present a graphical alert as a notification or, in other words, status message format. That is, patient device 24 may present notification 502 as shown in user interface 500A of the example in FIG. 5A.

Notification 502 indicates that projected levels of glucose may exceed the upper threshold of the prescribed range at some point in the future. As such, notification 502 includes a title “High Predicted” followed by a statement “I noticed a sudden rise.” As such, notification 502 presents a high glucose alert warning that patient 12 may experience a hyperglycemic event. In this instance, patient 12 may have missed a bolus, where patient 12 may interact with (e.g., select) notification 502 to expand notification 502 (which includes additional information as shown by the three dots “. . . ” in the example of FIG. 5A).

Referring next to the example of FIG. 5B, patient 12 has selected notification 502, expanding notification 502 into a notification 504 shown in a user interface 500B of the user interface presented by patient device 24. Notification 504 includes the same title as notification 502 along with the same statement “I noticed a sudden rise.” Notification 504 further includes, however, an additional statement regarding carbohydrate consumption that indicates that it “Looks like you had around 20 g of carb without dosing,” followed by the question “Is that right?” Notification 504 also includes two interface elements 506 and 508, e.g., touchscreen interface elements, with which patient 12 can interface to specify a respective answer of “Yes” or “No” to the above question. In the example of FIG. 5B, it is assumed that patient 12 selects interface element 506 to answer “Yes,” transitioning the user interface of patient device 24 to the user interface shown in the example of FIG. SC,

In the example of FIG. 5C, user interface 500C presented by patient device 24 includes a graph similar to the graph shown in the example of FIG. 4A. The graph includes a current glucose level 404 of “125” along with past glucose levels indicated by line 405. The graph also includes prescribed range 406 delineated by lower threshold 408 and upper threshold 410. The graph may facilitate understanding by patient 12 of the current glucose levels and allow patient 12. to determine whether to deliver one or more units of insulin.

As further shown by user interface 5000, the user interface may also acknowledge that patient 12 has selected interface element 506 to indicate that approximately 20 grams of carbohydrates were consumed, where the user interface indicates “Got it, If you dose 2 u [units] of insulin you should come back in range.” As such, patient device 24 may recommend a dosage of 2 units of insulin to bring patient 12 back within prescribed range 406, as currently projected levels of glucose determined under the assumption that patient 12 has consumed 20 grams of carbohydrates may exceed upper threshold 410.

To facilitate delivery of insulin, the user interface presented in user interface 500C may also include two interface elements 510 and 512 that allow patient 12 to indicate “Not yet” and “Ok let's do it,” respectively. Responsive to patient 12 interfacing with interface element 510, patient device 24 may refrain from interfacing with insulin pump 14 to deliver two units of insulin. Responsive to patient 12 interfacing with interface element 512, patient device 24 may interface with insulin pump 14 to cause the insulin pump to deliver two units of insulin.

In this respect, the examples shown in FIGS. 5A-5C may enable patient device 24 to monitor whether patient 12 has forgotten to take a bolus and, responsive to detecting that patient 12 has forgotten to take a bolus, generate a recommendation to avoid excessive high glucose levels. Moreover, patient device 24, in performing the foregoing operations, increases adherence by patient 12 to log insulin delivery amounts in case patient 12 forgets to enter such insulin delivery amounts in patient device 24, Such logging may in turn help any insulin dosing algorithms (and corresponding physicians or other care providers) to calculate a correct insulin dose and settings for achieving better control.

FIGS, 6A-6C are diagrams illustrating user interfaces of the patient device discussed with respect to the examples of FIGS. 1-3 in automatically switching between projection modes according to various aspects of the techniques described in this disclosure. In the example of FIG. 6A, the user interface presented by a user interface 600A provides a graph depicting projected levels of glucose 602 according to a first projection mode (i.e., a 2 hour projection mode in this example as denoted by the “+2”). Patient device 24 may interface with sensor 20 to obtain current glucose level 604 and determine, based on current glucose level 604, projected levels of glucose 602. The graph shown in user interface 600A also includes prescribed range 606 having a lower threshold 608 and an upper threshold 610.

The user interface depicted by user interface 600A shows a short term glucose projection in which projected levels of glucose are shown over a two hour time frame. The graph includes a time stamp at the top that represents the corresponding time. In some examples, the plot can be optional where patient 12 may decide to bring up such projected levels of glucose 602 by interacting with the user interface to slide the plot to the left.

In any event, patient device 24 may determine an occurrence of a projection event, such as a meal event, that alters projection of glucose levels. For example, assume patient 12 consumes a meal, where wearable device 22 may provide movements to patient device 24, which analyzes the movements to automatically determine whether or not patient 12 has begun consumption of a meal. In this example, patient device 24 may determine, based on the movements (or data indicative thereof), that patient 12 has begun consuming a meal. As such, patient device 24 may automatically determine that a projection event has occurred that alters how projected levels of glucose 602 is to be output. Patient device 24 may then update the user interface to show updated projected levels of glucose over a different time frame.

In the example of FIG. 6B, patient device 24 has detected the projection event and updated the user interface as shown in user interface 600B. The user interface shown in user interface 600B provides a graph depicting projected levels of glucose 612 over a four hour time frame (as denoted by the “+4”). Patient device 24 may detect that the meal projection event has occurred at approximately 11:30 AM. Based on classifying the projection event as a meal event, patient device 24 may automatically determine a second time frame (i.e., the four hour time frame in this example). Patient device 24 may interface with sensor 20 to obtain a current glucose level 614 of patient 12. Based on current glucose level 614. patient device 24 may obtain projected levels of glucose 612 in patient 12 over this four hour time frame. Patient device 24 may output the graph shown in user interface 600B that shows projected levels of glucose 612 over the four hour time frame.

Patient device 24 may denote the projection event using icons 616 and 618. Icon 616 denotes that patient 12 has performed a projection event (as icon 616 is a depiction of a person). Icon 618 denotes that patient 12 has eaten a meal (as icon 618 is a depiction of a fork and knife), Patient device 24 may automatically determine the four hour time frame over which to project glucose levels for meal events, insulin delivery events, and/or exercise events.

In any event, as time passes, patient device 24 may determine that patient 12 is about to no to sleep (e.g., a time of day event in which patient device 24 may determine that patient 12 routinely sleeps around 11 PM). That is, patient device 24 may determine, based on a current time (i.e., 11 PM in the example of FIG. 6C), that patient 12 routinely goes to sleep shortly after 11 PM. Patient device 24 may automatically determine, based on this sleep projection event, a third time frame over which to project glucose levels (e.g., an eight hour time frame). Patient device 24 may then update the user interface to reflect this new projection.

In the example of FIG. 6C, user interface600C shows projected levels of glucose 622 over the eight hour time frame (as denoted in the graph by “+8”). Patient device 24 may interface with sensor 20 to obtain a current glucose level 624, and determine, based on current glucose level 624, projected levels of glucose 622 over the eight hour time frame. Patient device 24 may further determine that patient 12 has consumed a meal (such as a small meal, which may also be referred to as a snack), updating the graph shown in user interface 600C with icons 626 and 628 to denote (similar to the example of FIG, 6B) consumption of a meal.

As further shown in the example of FIG. 6C, patient device 24 may update the graph to denote future expected behavior. In user interface 600C, patient device 24 presents the graph with additional icons 636 and 638, which denote that patient 12 regularly consumes a meal at approximately 6:30 AM in the morning following the sleep projection event. As such, projected levels of glucose 622 are expected to rise after consumption of the meal at approximately 6:30 AM. Patient device 24 may update graph to reflect other types of projection events, such as exercise events, insulin delivery events, etc, or otherwise recommend various actions to mitigate or otherwise manage possible hypoglycemic events or hyperglycemic events.

By dynamically switching between different projection modes, patient device 24 may allow patient 12 to quickly identify any issues with glucose levels and take the appropriate action without having to manually indicate each mode. Moreover, rather than default to a particular prediction mode that may not suit the current context, patient device 24 may dynamically switch between projection modes to potentially accommodate the current context and enable patient 12 to better understand how to manage glucose levels.

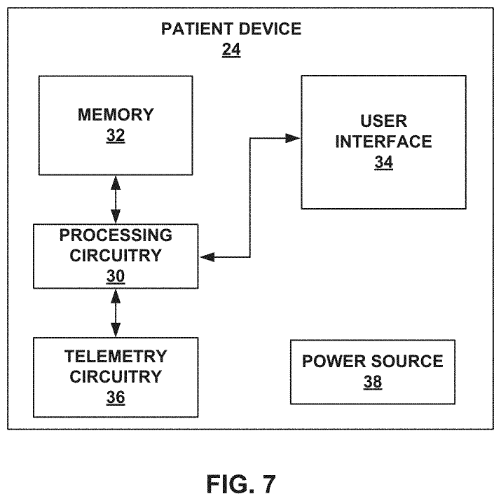

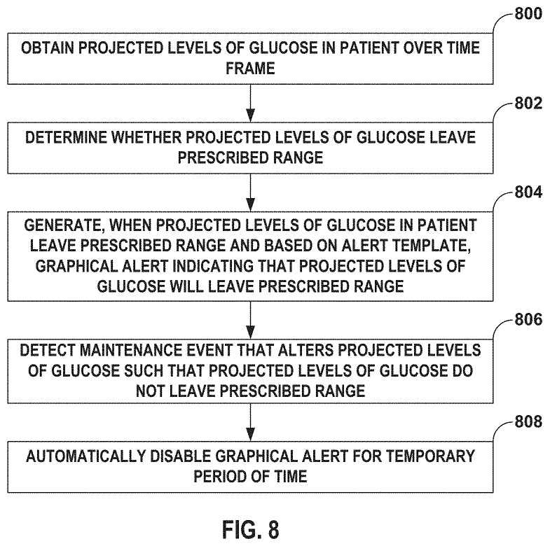

FIG. 7 is a block diagram illustrating an example of a patient device, in accordance with one or more examples described in this disclosure. While patient device 24 may generally be described as a hand-held computing device, patient device 24 may be a notebook computer, a cell phone, or a workstation, for example. In some examples, patient device 24 may be a mobile device, such as a smartphone or a tablet computer. In such examples, patient device 24 may execute an application that allows patient device 24 to perform example techniques described in this disclosure. In some examples, patient device 24 may be specialized controller for communicating with insulin pump 14.

Although the examples are described with one patient device 24, in some examples, patient device 24 may be a combination of different devices (e.g., mobile device and a controller). For instance, the mobile device may provide access to one or more processors 28 of cloud 26 through Wi-Fi or carrier network and the controller may provide access to insulin pump 14. In such examples, the mobile device and the controller may communicate with one another through Bluetooth. Various combinations of a mobile device and a controller together forming patient device 24 are possible and the example techniques should not be considered limited to any one particular configuration.

As illustrated in FIG. 7, patient device 24 may include a processing circuitry 30, memory 32, user interface 34, telemetry circuitry 36, and power source 38. Memory 32 may store program instructions that, when executed by processing circuitry 30, cause processing circuitry 30 to provide the functionality ascribed to patient device 24 throughout this disclosure.

In some examples, memory 32 of patient device 24 may store a plurality of parameters, such as amounts of insulin to deliver, target glucose level, time of delivery etc. Processing circuitry 30 through telemetry circuitry 36) may output the parameters stored in memory 32 to insulin pump 14 or injection device 30 for delivery of insulin to patient 12. In examples, processing circuitry 30 may execute a notification application, stored in memory 32, that outputs notifications to patient 12, such as notification to take insulin, amount of insulin, and time to take the insulin, via user interface 34.

Memory 32 may include any volatile, non-volatile, fixed, removable, magnetic, optical, or electrical media, such as RAM, ROM, hard disk, removable magnetic disk, memory cards or sticks, EEPROM, flash memory, and the like. Processing circuitry 30 can take the form one or more microprocessors, DSPs, ASICs, FPGAs, programmable logic circuitry, or the like, and the functions attributed to processing circuitry 30 herein may be embodied as hardware, firmware, software or any combination thereof.

User interface 34 may include a button or keypad, lights, a speaker for voice commands, and a display, such as a liquid crystal (LCD) a light emitting diode (LED) display, an organic LED (OLED) display, etc. In some examples the display may be a presence-sensitive display. As discussed in this disclosure, processing circuitry 30 may present and receive information relating to therapy via user interface 34. For example, processing circuitry 30 may receive patient input via user interface 34. The patient input may be entered, for example, by pressing a button on a keypad, entering text, or selecting an icon from a touch screen. The patient input may be information indicative of food that patient 12 eats, such as for the initial learning phase, whether patient 12 took the insulin (e.g., through the syringe or injection device 30), and other such information.

Telemetry circuitry 36 includes any suitable hardware, firmware, software or any combination thereof for communicating with another device, such as cloud 26, insulin pump 16 or injection device 30, as applicable, wearable device 22, and sensor 20. Telemetry circuitry 36 may receive communication with the aid of an antenna, which may be internal and/or external to patient device 24. Telemetry circuitry 36 may be configured to communicate with another computing device via wireless communication techniques, or direct communication through a wired connection. Examples of local wireless communication techniques that may be employed to facilitate communication between patient device 24 and another computing device include RF communication according to IEEE 802.11 or Bluetooth specification sets, infrared communication, e.g., according to an IrDA standard, or other standard or proprietary telemetry protocols. Telemetry circuitry 36 may also provide connection with carrier network for access to cloud 26. In this manner, other devices may be capable of communicating with patient device 24.

Power source 38 delivers operating power to the components of patient device 24. In some examples, power source 38 may include a battery, such as a rechargeable or non-rechargeable battery. A non-rechargeable battery may be selected to last for several years, while a rechargeable battery may be inductively charged from an external device, e.g., on a daily or weekly basis. Recharging of a rechargeable battery may be accomplished by using an alternating current (AC) outlet or through proximal inductive interaction between an external charger and an inductive charging coil within patient device 24.

Processing circuitry 30 may interface with telemetry circuitry 36 to communicate with sensor 20, whereby processing circuitry 30 may obtain a current glucose level sensed by sensor 20 in patient 12. Processing circuitry 30 may determine, based on the current glucose level, the projected levels of glucose in patient 12 over a time frame. Processing circuitry 30 may determine whether the projected levels of glucose leave a prescribed range. Processing circuitry 30 may generate, when the projected levels of glucose in patient 12 leave the prescribed range and based on an alert template (which may be stored to memory 32), a graphical alert indicating that the projected levels of glucose will leave the prescribed range (as shown in the examples of FIGS. 4A and 4B).

Processing circuitry 30 may interface with user interface 34 to output the graphical alert, examples of which may be shown by way of user interfaces 400A and 400B in FIGS. 4A and 4B. Processing circuitry 30 may then automatically detect a maintenance event that alters the projected levels of glucose such that the projected levels of glucose do not leave the prescribed range. Processing circuitry 30 may detect the maintenance event and interface with telemetry circuit 36 to communicate with sensor 20 to once again obtain a current glucose level sensed by sensor 20. Based on the current glucose level, processing circuitry 30 may determine updated projected levels of glucose. Processing circuitry 30 may determine that the updated projected levels of glucose do not leave the prescribed range.

Responsive to determining that the updated projected levels of glucose do not leave the prescribed range, processing circuitry 30 may automatically disable the graphical alert for a temporary period of time. Processing circuitry 30 may automatically set the temporary period of time based on an expected duration until the previously projected levels of glucose were to leave the prescribed range as described above.

In addition, processing circuitry 30 may determine an occurrence of a projection event that alters how the projected levels of glucose are to be output. Projection events may include tune of day events, such as sleep and/or nap tune for patient 12, a time at which patient 12 wakes up, and a time at which patient 12 eats a meal (including snacks or other meal events). Projection events may also include physiological events, such as hyperglycemic events, hypoglycemic events, illness experienced by patient 12, menstrual cycle of patient 12, consumption of new and/or changing medications regimens, etc.

Further, projection events may include lifestyle events, such as user preferences and/or settings, unavailability of patient 12 (e.g., due to long meetings, traveling and/or airplane mode, and/or other do not disturb events), vacations, holidays or other social events, sedentary versus active lifestyles, small versus large meals or boluses. Projection events may, additionally, include data driven events, such as missing, uncertain and/or inaccurate data inputs, recent prediction inaccuracy, historical patient-specific prediction inaccuracy, and/or event detection via a connected devices, such as wearable device 22 and/or an insulin pen and/or insulin pump.

In any event, processing circuitry 30 may automatically determine, based on the projection event, a different time frame over which to project levels of glucose. As such, processing circuitry 30 may automatically switch between projection modes based on the projection event, which accounts for the current context in which patient 12 is operating. Processing circuitry 30 may again interface with telemetry circuit 36 to communicate with sensor 20 to obtain the current glucose level, Based on the current glucose level, processing circuitry 30 may determine projected levels of glucose over the different time frame. Processing circuitry 30 may interface with user interface 34 to output a user interface that includes the projected levels of glucose over the different time frame (such as that shown by user interfaces 600A-600C in the examples of FIG. 6A-6C).

For example, processing circuitry 30 may automatically detect a meal event (possibly by analyzing movements sensed by wearable device 22) indicating that patient 12 is currently eating a meal. Processing circuitry 30 may automatically determine, based on the meal event, the different time frame as a four hour time frame, switching projection modes from the shorter two hour time frame to the longer four hour time frame. In some instances, processing circuitry 30 may prompt the user to enter a size of the meal, as a smaller meal or snack may result in processing circuitry 30 refraining from switching from the shorter two hour time frame.

Processing circuitry 30 may also, as another example, interface with telemetry circuitry 36 to communicate with insulin pump 14 in order to automatically detect an insulin delivery event indicating that patient 12 has received insulin. Processing circuitry 30 may then automatically (meaning without or with very limited input from patient 12) determine, based on the insulin delivery event, the four hour time frame, switching projection modes from either the longer eight hour time frame or from the shorter two hour time frame,

Processing circuitry 30 may also determine, as a further example, an exercise event indicating that patient 12 is currently exercising. Processing circuitry 30 may interface with telemetry circuitry 36 to communicate with wearable device 22 in order to receive data indicative of a heart rate, blood-oxygen level, respiration rate, and the like, which may be used to determine the exercise event. Processing circuitry 30 may also obtain global positioning system coordinates of patient 12, accelerometer data from an accelerometer within patient device 24, or other data commonly used to identify different exercise activities. Processing circuitry 30 may then determine, based on the exercise event, the different time frame.

Additionally, processing circuitry 30 may automatically detect a sleep event indicating that patient 12 is expected to sleep. Processing circuitry 30 may interface with telemetry circuitry 36 to communicate with wearable device 22 in order to receive data indicative of a heart rate, blood-oxygen level, respiration rate, and the like, which may be used to automatically detect the sleep event. Processing circuitry 30 may also obtain global positioning system coordinates of patient 12, accelerometer data from an accelerometer within patient device 24, or other data commonly used to identify sleep activities. Processing circuitry 30 may then determine, based on the sleep event, the eight hour time frame.

FIG, 8 is a flowchart illustrating example operation of the patient device shown in FIGS. 1-3 and 7 in performing various aspects of the automated alert disabling techniques. Processing circuitry 30 of patient device 24 (shown in the example of FIG. 7) may interface with telemetry circuitry 36 to communicate with sensor 20, whereby processing circuitry 30 may obtain a current glucose level in patient 12. Processing circuitry 30 may dete3mine, based on the current glucose level, the projected levels of glucose in patient 12 over a time frame (800). Processing circuitry 30 may determine whether the projected levels of glucose leave a prescribed range (802). Processing circuitry 30 may generate, when the projected levels of glucose in patient 12 leave the prescribed range and based on an alert template (which may be stored to memory 32), a graphical alert indicating that the projected levels of glucose will leave the prescribed range (as shown in the examples of FIGS. 4A and 4B) (804).

Processing circuitry 30 may interface with user interface 34 to output the graphical alert, examples of which may be shown by way of user interfaces 400A and 400B in FIGS. 4A and 4B. Processing circuitry 30 may then automatically detect a maintenance event that alters the projected levels of glucose such that the projected levels of glucose do not leave the prescribed range (806). Processing circuitry 30 may detect the maintenance event and interface with telemetry circuit 36 to communicate with sensor 20 to once again obtain a current glucose level. Based on the current glucose level, processing circuitry 30 may determine updated projected levels of glucose. Processing circuitry 30 may determine that the updated projected levels of glucose do not leave the prescribed range.

Responsive to determining that the updated projected levels of glucose do not leave the prescribed range, processing circuitry 30 may automatically disable the graphical alert for a temporary period of time (806). Processing circuitry 30 may automatically set the temporary period of time based on an expected duration until the previous projected levels of glucose were to leave the prescribed range as described above.

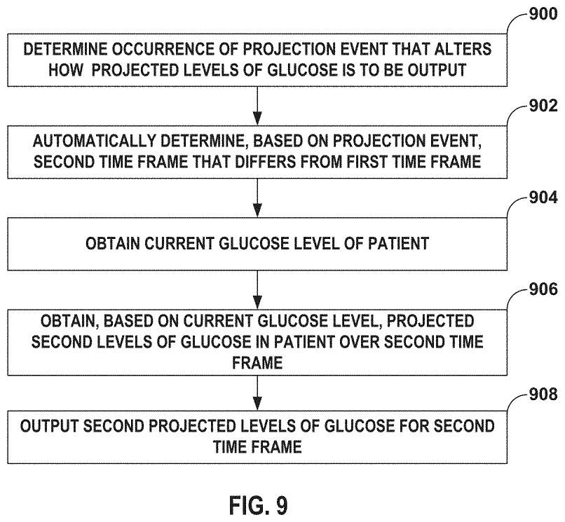

FIG. 9 is a flowchart illustrating example operation of the patient device shown in FIGS, 1-3 and 7 in performing various aspects of the automated projection mode switching techniques. Processing circuitry 30 may determine an occurrence of a projection event that alters how the projected levels of glucose are to be output (900). Processing circuitry 30 may automatically determine, based on the projection event, a. different time frame over which to project levels of glucose (902). As such, processing circuitry 30 may automatically switch between projection modes based on the projection event, which accounts for the current context in which patient 12 is operating.

Processing circuitry 30 may again interface with telemetry circuit 36 to communicate with sensor 20 to obtain the current glucose level (904). Based on the current glucose level, processing circuitry 30 may determine projected levels of glucose over the different time frame (906). Processing circuitry 30 may interface with user interface 34 to output a user interface that includes the projected levels of glucose over the different time frame (such as that shown by user interfaces 600A-600C in the examples of FIG. 6A-6C) (908).

In this way, various aspects of the techniques may enable the following examples.

Example 1. A device for assisting in therapy delivery, the device comprising: a memory configured to store alert data; and one or more processors configured to: obtain projected levels of glucose in a patient over a time frame; determine whether the projected levels of glucose leave a prescribed range; generate, when the projected levels of glucose in the patient leave the prescribed range during the time frame and based on the alert data, a graphical alert indicating that the projected levels of glucose will leave the prescribed range; determine that a maintenance event alters the projected levels of glucose such that the projected levels of glucose do not leave the prescribed range; and disable, without user input and based on the determination that the maintenance event alters the projected levels of glucose such that the projected levels of glucose do not leave the prescribed range, the graphical alert for a temporary period of time.

Example 2. The device of example 1, wherein the one or more processors are configured to: obtain, from a glucose sensor, a current level of glucose in the patient; and obtain, based on the current level of glucose, the projected levels of glucose in the patient over the time frame.

Example 3. The device of any combination of examples 1 and 2, wherein the one or more processors are, when determining that the maintenance event alters the projected levels of glucose, configured to: automatically detect a meal event indicating that the patient is currently eating a meal; obtain, based on the meal event, a revised version of the projected levels of glucose; and determine that the revised version of the projected levels of glucose do not leave the prescribed range.

Example 4. The device of example 3, wherein the one or more processors are configured to: interface with a wearable computing device worn by the patient to identify movements performed by the patient; and automatically detect, based on the movements, the meal event indicating that the patient is currently eating a meal.

Example 5. The device of any combination of examples 1-4, wherein the one or more processors are, when determining that the maintenance event alters the projected levels of glucose, configured to: automatically detect an insulin delivery event indicating that the patient has injected insulin; obtain, based on the insulin delivery event, a revised version of the projected levels of glucose; and determine that the revised version of the projected levels of glucose do not leave the prescribed range.

Example 6. The device of any combination of examples 1-5, wherein the maintenance event is a first maintenance event, and wherein the One or more processors are further configured to: detect initiation of a second maintenance event; determine an amount associated with the second maintenance event; and determine, based on the amount associated with the second maintenance event, that the projected levels of glucose will leave the prescribed range.

Example 7. The device of claim 6, wherein the one or more processors are further configured to: present the graphical alert along with an audible alert to the patient; and present, responsive to determining that the projected levels of glucose will leave the prescribed range, a haptic alert in place of the audible alert such that the audible alert is disabled for the temporary period of time.

Example 8. The device of any combination of examples 1-7, wherein the one or more processors are further configured to: determine a duration until the projected levels of glucose are projected to leave the prescribed range; and determine, based on the duration, the temporary period of time by which to automatically disable the graphical alert.

Example 9. The device of any combination of examples 1-8, wherein the graphical alert further includes a prompt for a user to disable the graphical alert for the temporary period of time via user input.

Example 10. The device of example 9, wherein the one or more processors are configured to: determine a duration until the projected levels of glucose are projected to leave the prescribed range; and determine, based on the duration, the temporary period of time by which to automatically disable the graphical alert.

Example 11. The device of any combination of examples 1-10, wherein the prescribed range includes values between a lower threshold identifying hypoglycemic condition for the patient and an upper threshold identifying a hyperglycemic condition for the patient.

Example 12. A method for assisting in therapy delivery, the method comprising: obtaining, by one or more processors, projected levels of glucose in a patient over a time frame; determining, by the one or more processors, whether the projected levels of glucose leave a prescribed range; generating, by the one or more processors, when the projected levels of glucose in the patient leave the prescribed range, and based on alert data, a graphical alert indicating that the projected levels of glucose will leave the prescribed range; determine, by the one or more processors, that a maintenance event alters the projected levels of glucose such that the projected levels of glucose do not leave the prescribed range; and automatically disabling, by the one or more processors and based on the determination that the maintenance event alters the projected levels of glucose such that the projected levels of glucose do not leave the prescribed range, the graphical alert for a temporary period of time.

Example 13. The method of example 12, wherein obtaining the projected levels of glucose comprises: obtaining, from a glucose sensor, a current level of glucose in the patient; and obtaining, based on the current level of glucose, the projected levels of glucose in the patient over the time frame.

Example 14. The method of any combination of examples 12 and 13, wherein determining that the maintenance event alters the projected levels of glucose comprises: automatically detecting a meal event indicating that the patient is currently eating a meal; obtaining, based on the meal event, a revised version of the projected levels of glucose; and determining that the revised version of the projected levels of glucose do not leave the prescribed range.

Example 15. The method of example 14, wherein automatically detecting the meal event comprises: interfacing with a wearable computing device worn by the patient to identify movements performed by the patient; and automatically detecting, based on the movements, the meal event indicating that the patient is currently eating a meal,

Example 16. The method of any combination of examples 12-15, wherein determining that the maintenance event alters the projected levels of glucose comprises: automatically detecting an insulin delivery event indicating that the patient has injected insulin; obtaining, based on the insulin delivery event, a revised version of the projected levels of glucose; and determining that the revised version of the projected levels of glucose do not leave the prescribed range.

Example 17. The method of any combination of examples 12-16, wherein the maintenance event is a first maintenance event and wherein the method further comprises: detecting initiation of a second maintenance event; determining an amount associated with the second maintenance event; and determining, based on the amount associated with the second maintenance event, that the projected levels of glucose will leave the prescribed range.

Example 18. The method of example 17, further comprising: presenting the graphical alert along with an audible alert to the patient, presenting, responsive to determining that the projected levels of glucose will leave the prescribed range, a haptic alert in place of the audible alert such that the audible alert is disabled for the temporary period of time.

Example 19. The method of any combination of examples 12-18, further comprising: determining a duration until the projected levels of glucose are projected to leave the prescribed range; and determining, based on the duration, the temporary period of time by which to automatically disable the graphical alert.

Example 20. A non-transitory computer-readable storage medium having instructions stored thereon that, when executed, cause one or more processors to: obtain projected levels of glucose in a patient over a time frame; determine whether the projected levels of glucose leave a prescribed range; generate, when the projected levels of glucose in the patient leave the prescribed range and based on an alert template, a graphical alert indicating that the projected levels of glucose will leave the prescribed range; determine that a maintenance event alters the projected levels of glucose such that the projected levels of glucose do not leave the prescribed range; and automatically disable, based on the determination that the maintenance event alters the projected levels of glucose such that the projected levels of glucose do not leave the prescribed range, the graphical alert for a temporary period of time,

Example 21. A device for assisting in therapy delivery, the device comprising: a memory configured to store first projected levels of glucose in a patient over a first time frame one or more processors configured to: determine an occurrence of a projection event that alters how projected levels of glucose are to be output; automatically determine, based on the projection event, a second time frame that differs from the first time frame; obtain a current glucose level of the patient; obtain, based on the current glucose level, second projected levels of glucose in the patient over the second time frame; and output the second projected levels of glucose for the second time frame.

Example 22. The device of example 21, wherein the projection event comprises one or more of a time of day event, a physiological event, a lifestyle event, or a data-driven. event.

Example 23. The device of any combination of examples 21 and 22, wherein the one or more processors are further configured to: determine whether the second projected levels of glucose leave a prescribed range; generate, responsive to determining that the second projected levels of glucose leave the prescribed range, a graphical alert indicating that the second projected levels of glucose will leave the prescribed range during the second time frame; and output the alert.

Example 24. The device of example 23, wherein the prescribed range includes values between a lower threshold identifying a hypoglycemic condition for the patient and an upper threshold identifying a hyperglycemic condition for the patient.

Example 25. The device of any combination of examples 21-24, wherein the one or more processors are, when determining the occurrence of the projection event, configured to automatically detect a meal event indicating that the patient is currently eating a meal, and wherein the one or more processors are, when automatically determining the second time frame, configured to automatically determine, based on the meal event, the second time frame.

Example 26. The device of example 25, wherein the second time frame is a time frame of greater than or equal to two hours.

Example 27. The device of example 25, wherein the second time frame is a time frame of at least four hours.

Example 28. The device of example 25, wherein the one or more processors are configured to: interface with a wearable computing device worn by the patient to identify movements performed by the patient; and automatically detect, based on the movements, the meal event indicating that the patient is currently eating a meal.

Example 29. The device of examples 21-28, wherein the one or more processors are, when determining the occurrence of the projection event, configured to automatically detect an insulin delivery event indicating that the patient has received insulin, and wherein the one or more processors are, when automatically determining the second time frame, configured to automatically determine, based on the insulin delivery event, the second time frame.

Example 30. The device of any combination of examples 21-29, wherein the one or more processors are, when determining the occurrence of the projection event, configured to automatically detect an exercise event indicating that the patient is currently exercising, and wherein the one or more processors are, when automatically determining the second time frame, configured to automatically determine, based on the exercise event, the second time frame.

Example 31. The device of any combination of examples 21-30, wherein the one or more processors are, when determining the occurrence of the projection event, configured to automatically detect a sleep event indicating that the patient is expected to sleep, and wherein the one or more processors are, when automatically determining the second time frame, configured to automatically determine, based on the sleep event, the second time frame.

Example 32. The device of example 31. wherein the second time frame is a time frame of greater than or equal to four hours.

Example 33. The device of example 31, wherein the second time frame is a time frame of at least eight hours.

Example 34. The device of any combination of examples 21-33, wherein the projection event is a first projection event, and wherein the one or more processors are configured to: determine an occurrence of a second projection event that alters how the projected levels of glucose is to be output; automatically determine, based on the second projection event, a third time frame that differs from the first time frame and the second time frame; obtain the current glucose level of the patient; obtain, based on the current glucose level, projected third levels of glucose in the patient over the third time frame; and output the third projected levels of glucose for the third time frame.

Example 35. The device of any combination of examples 21-34, wherein the one or more processors are further configured to interface with a glucose monitor to obtain the current glucose level sensed by an insulin pump implanted in the patient.

Example 36. The device of any combination of examples 21-35, wherein the first time frame is for a first duration, and wherein the second time frame is for a second duration that is different than the first duration.

Example 37. A method for assisting in therapy delivery, the method comprising: obtaining, by one or more processors, first projected levels of glucose in a patient over a first time frame; determining, by the one or more processors, an occurrence of a projection event that alters how projected levels of glucose are to be output; automatically determining, by the one or more processors and based on the projection event, a second time frame that differs from the first time frame; obtaining, by the one or more processors, a current glucose level of the patient; obtaining, by the one or more processors and based on the current glucose level, second projected levels of glucose in the patient over the second time frame; and outputting, by the one or more processors, the second projected levels of glucose for the second time frame.

Example 38. The method of example 37, wherein the projection event comprises one or more of a time of day event, a physiological event, a lifestyle event, or a data-driven event.

Example 39. The method of any combination of examples 37 and 38, further comprising: determining whether the second projected levels of glucose leave a prescribed range; generating, responsive to determining that the second projected levels of glucose leave the prescribed range, a graphical alert indicating that the second projected levels of glucose will leave the prescribed range during the second time frame; and outputting the alert.

Example 40. A non-transitory computer-readable storage medium having instructions stored thereon that, when executed, cause one or more processors to: determine an occurrence of a projection event that alters how first projected levels of glucose are to be output; automatically determine, based on the projection event, a second time frame that differs from a first time frame over which the first projected levels of glucose were projected; obtain a current glucose level of the patient; obtain, based on the current glucose level, second projected levels of glucose in the patient over the second time frame, and output the second projected levels of glucose for the second time frame.

Various aspects of the techniques may be implemented within one or more processors, including one or more microprocessors, DSPs. ASICs, FPGAs, or any other equivalent integrated or discrete logic circuitry, as well as any combinations of such components, embodied in programmers, such as physician or patient programmers, electrical stimulators, or other devices, The term “processor” or “processing circuitry” may generally refer to any of the foregoing logic circuitry, alone or in combination with other logic circuitry, or any other equivalent circuitry.

In one or more examples, the functions described in this disclosure may be implemented in hardware, software, firmware, or any combination thereof. If implemented in software, the functions may be stored on, as one or more instructions or code, a computer-readable medium and executed by a hardware-based processing unit. Computer-readable media may include computer-readable storage media forming a tangible, non-transitory medium. Instructions may be executed by one or more processors, such as one or more DSPs, ASICs, FPGAs, general purpose microprocessors, or other equivalent integrated or discrete logic circuitry. Accordingly, the term “processor,” as used herein may refer to one or more of any of the foregoing structure or any other structure suitable for implementation of the techniques described herein.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including one or more processors 28 of cloud 26, one or more processors of patient device 24, one or more processors of wearable device 22, one or more processors of insulin pump 14, or some combination thereof. The one or more processors may be one or more integrated circuits (ICs), and/or discrete electrical circuitry, residing in various locations in the example systems described in this disclosure.

The one or more processors or processing circuitry (e.g., processors 28 (FIGS. 1-3) and processing circuitry 30 (FIG. 7)) utilized for example techniques described in this disclosure may be implemented as fixed-function circuits, programmable circuits, or a combination thereof. Fixed-function circuits refer to circuits that provide particular functionality, and are preset on the operations that can be performed. Programmable circuits refer to circuits that can be programmed to perform various tasks, and provide flexible functionality in the operations that can be performed. For instance, programmable circuits may execute software or firmware that cause the programmable circuits to operate in the manner defined by instructions of the software or firmware. Fixed-function circuits may execute software instructions e.g., to receive parameters or output parameters), but the types of operations that the fixed-function circuits perform are generally immutable. In Kale examples, the one or more of the units may be distinct circuit blocks (fixed-function or programmable), and in some examples, the one or more units may be integrated circuits. The processors or processing circuitry may include arithmetic logic units (ALUs), elementary function units (E Us). digital circuits, analog circuits, and/or programmable cores, formed from programmable circuits. In examples where the operations of the processors or processing circuitry are performed using software executed by the programmable circuits, memory accessible by the processors or processing circuitry may store the object code of the software that the processors or processing circuitry receive and execute.

Various aspects of the disclosure have been described. These and other aspects are within the scope of the following claims.

What is claimed is:

1. A device for assisting in therapy delivery, the device comprising:
a memory configured to store first projected levels of glucose in a patient over a first time frame:
one or more processors configured to:
determine an occurrence of a projection event that alters how projected levels of glucose are to be output;
automatically determine, based on the projection event, a second time frame that differs from the first time frame;
obtain a current glucose level of the patient;
obtain, based on the current glucose level, second projected levels of glucose in the patient over the second time frame; and
output the second projected levels of glucose for the second time frame.

2. The device of claim 1, wherein the projection event comprises one or more of a time of day event, a physiological event, a lifestyle event, or a data-driven event.

3. The device of claim 1, wherein the one or more processors are further configured to:
determine whether the second projected levels of glucose leave a prescribed range;
generate, responsive to determining that the second projected levels of glucose leave the prescribed range, a graphical alert indicating that the second projected levels of glucose will leave the prescribed range during the second time frame; and
output the alert.

4. The device of claim 3, wherein the prescribed range includes values between a lower threshold identifying a hypoglycemic condition for the patient and an upper threshold identifying a hyperglycemic condition for the patient.

5. The device of claim 1,
wherein the one or more processors are, when determining the occurrence of the projection event, configured to automatically detect a meal event indicating that the patient is currently eating a meal, and
wherein the one or more processors are, when automatically determining the second time frame, configured to automatically determine, based on the meal event, the second time frame.

6. The device of claim 5, wherein the second time frame is a time frame of greater than or equal to two hours.

7. The device of claim 5, wherein the second time frame is a time frame of at least four hours.

8. The device of claim 5, wherein the one or more processors are configured to:
interface with a wearable computing device worn by the patient to identify movements performed by the patient; and
automatically detect, based on the movements, the meal event indicating that the patient is currently eating a meal.

9. The device of claim 1,
wherein the one or more processors are, when determining the occurrence of the projection event, configured to automatically detect an insulin delivery event indicating that the patient has received insulin, and
wherein the one or more processors are, when automatically determining the second time frame, configured to automatically determine, based on the insulin delivery event, the second time frame.

10. The device of claim 1,
wherein the one or more processors are, when determining the occurrence of the projection event, configured to automatically detect an exercise event indicating that the patient is currently exercising, and
wherein the one or more processors are, when automatically determining the second time frame, configured to automatically determine, based on the exercise event, the second time frame.

11. The device of claim 1,
wherein the one or more processors are, when determining the occurrence of the projection event, configured to automatically detect a sleep event indicating that the patient is expected to sleep, and
wherein the one or more processors are, when automatically dete3mining the second time frame, configured to automatically determine, based on the sleep event, the second time frame.

12. The device of claim 11. wherein the second time frame is a time frame of greater than or equal to four hours.

13. The device of claim 11, wherein the second time frame is a time frame of at least eight hours.

14. The device of claim 1,
wherein the projection event is a first projection event, and
wherein the one or more processors are configured to:
determine an occurrence of a second projection event that alters how the projected levels of glucose is to be output;
automatically determine, based on the second projection event, a third time frame that differs from the first time frame and the second time frame;
obtain the current glucose level of the patient;
obtain, based on the current glucose level, projected third levels of glucose in the patient over the third time frame; and
output the third projected levels of glucose for the third time frame.

15. The device of claim 1, wherein the one or more processors are further configured to interface with a glucose monitor to obtain the current glucose level sensed by an insulin pump implanted in the patient.

16. The device of claim 1,
wherein the first time frame is for a first duration, and
wherein the second time frame is for a second duration that is different than the first duration.

17. A method for assisting in therapy delivery, the method comprising:
obtaining, by one or more processors, first projected levels of glucose in a patient over a first time frame;
determining, by the one or more processors, an occurrence of a projection event that alters how projected levels of glucose are to be output;
automatically determining, by the one or more processors and based on the projection event, a second time frame that differs from the first time frame;
obtaining, by the one or more processors, a current glucose level of the patient;
obtaining, by the one or more processors and based on the current glucose level, second projected levels of glucose in the patient over the second time frame; and
outputting, by the one or more processors, the second projected levels of glucose for the second time frame.

18. The method of claim 17, wherein the projection event comprises one or more of a time of day event, a physiological event, a lifestyle event, or a data-driven event.

19. The method of claim 17, further comprising:
determining whether the second projected levels of glucose leave a prescribed range;
generating, responsive to determining that the second projected levels of glucose leave the prescribed range, a graphical alert indicating that the second projected levels of glucose will leave the prescribed range during the second time frame; and
outputting the alert.

20. A non-transitory computer-readable storage medium having instructions stored thereon that, when executed, cause one or more processors to:
determine an occurrence of a projection event that alters how first projected levels of glucose are to be output;
automatically determine, based on the projection event, a second time frame that differs from a first time frame over which the first projected levels of glucose were projected;
obtain a current glucose level of the patient;
obtain, based on the current glucose level, second projected levels of glucose in the patient over the second time frame; and
output the second projected levels of glucose for the second time frame.
